(19) United States
(12) Patent Application Publication
(10) Pub. No.: US 2025/0001160 A1
(43) Pub. Date: Jan. 2, 2025

(54) DEVICES AND METHODS FOR DELIVERING BLOOD FROM A LOWER PRESSURE REGION TO A HIGHER PRESSURE REGION

(71) Applicant: CARDIOST, INC., Houston, TX (US)

(72) Inventors: Fernando ANZELLINI, Houston, TX (US); Nicolás ANZELLINI, Houston, TX (US)

(21) Appl. No.: 18/818,358

(22) Filed: Aug. 28, 2024

Related U.S. Application Data
(63) Continuation of application No. PCT/US2023/063511, filed on Mar. 1, 2023.
(60) Provisional application No. 63/315,700, filed on Mar. 2, 2022.

(51) Int. Cl. A61M 60/857; A61M 60/122; A61M 60/205; A61M 60/247; A61M 60/50; A61M 60/873

(52) U.S. Cl. CPC A61M 60/857; A61M 60/122; A61M 60/205; A61M 60/247; A61M 60/50; A61M 60/873; A61M 2205/04; A61M 2205/103; A61M 2205/106; A61M 2205/3334; A61M 2205/3515; A61M 2205/3523; A61M 2205/50; A61M 2205/8206; A61M 2206/20; A61M 2210/127

(57) ABSTRACT

A device and method for diverting a portion of oxygenated blood from a lower pressure region, e.g., left atrium or pulmonary vein, and providing it to the aorta, bypassing the left ventricle, operating at least in part, on the Venturi effect are disclosed herein. The device includes a first conduit disposed within the aorta. The device includes a second conduit that delivers blood from the lower pressure region to the first conduit. The blood from the lower pressure region in the second conduit is combined with the blood from the aorta in the first conduit. The second conduit is coupled to the first conduit at or near a narrow segment of the first conduit. A pump may pump blood along the second conduit in some examples. A Venturi effect at or near the narrow segment draws the blood from the lower pressure region into the first and/or second conduit.

### RELATED APPLICATIONS

Tis application is the by-pass continuation of PCT International Application No. PCT/US2023/063511, filed Mar. 1, 2023, which claims priority to U.S. Provisional Application No. 63/315,700, filed Mar. 2, 2022, the entire contents of each are incorporated herein in their entirety.

### TECHNICAL FIELD

The present disclosure pertains to devices and methods for delivering blood from a lower pressure region to a higher pressure region. In particular, the present disclosure pertains to devices and methods for delivering oxygenated blood from a chamber of a heart to a blood vessel.

### BACKGROUND

The left ventricle of the heart is responsible for pumping oxygenated blood received from the lungs to the aorta to deliver the oxygenated blood to the rest of the body. The left ventricle receives oxygenated blood from the lungs via the left atrium (auricle). The left ventricle of a healthy heart has an ejection fraction of approximately 50 to 70 percent. In other words, with each heartbeat, the left ventricle provides 50 to 70 percent of blood received from the left atrium to the aorta. When the ejection fraction of the left ventricle falls below 50 percent, the heart is no longer efficiently providing oxygenated blood to the rest of the body. Reduced ejection fraction (HFrEF), medium ejection fraction (HFmEF), and preserved ejection fraction (HFpEF) that are symptomatic of heart failure may be associated with one or more diseases or disorders such as congenital heart defects, cardiomyopathy, diabetes, coronary artery disease, myocardial infarction, and uncontrolled high blood pressure. Subjects suffering from heart failure with reduced, medium, or preserved ejection fractions may suffer health effects such as fatigue, weakness, edema, shortness of breath, and mental confusion. Depending on the severity, heart failure with reduced, medium, and preserved ejection fraction may lead to reduced quality of life or death if left untreated.

In some cases, the underlying cause of heart failure with reduced, medium, or preserved ejection fraction of the left ventricle can be corrected, for example, by repairing or replacing a malfunctioning mitral valve. However, in other cases, such as when the cardiac muscle of the ventricle is weakened or damaged (e.g., stiffened and/or thickened walls), the cause of the reduced, medium, or preserved ejection fraction cannot be corrected or reversed. In these instances, particularly for severe cases, a subject may require a heart transplant, an artificial heart, or an interventional device. The availability of donated hearts for transplants is very low, recipients are required to remain on immunosuppressant drugs with varying levels of side effects, and even with these drugs, there is still the potential for organ rejection by the recipient.

Mechanical and electromechanical devices are available such as atrial shunts, left ventricle expanders, electrical and neurostimulators, and mechanical circulatory support. However, these existing devices are often only effective short term, cause long-term detrimental effects, are better suited to reduced-ejection fraction forms of heart failure, and/or require a complex device. Accordingly, improved techniques for compensating for preserved ejection fraction of the left ventricle are desired.

### SUMMARY

Devices and methods are disclosed for delivering blood from a lower pressure region to a higher pressure region.

According to at least one embodiment of the disclosure, a device for delivering blood from a lower pressure region to a higher pressure region may include a first conduit configured to be disposed within the higher pressure region, and the conduit may include a first end having a first diameter, a second end opposite the first end and having a second diameter, and a narrow segment disposed between the first end and the second end and having a third diameter such that the third diameter is less than the first diameter and the second diameter. The device may also include a second conduit having a third end configured to be fluidly coupled to the lower pressure region, and a fourth end opposite the third end, such that the fourth end is coupled to the first conduit at or near the narrow segment of the first conduit. The device may further include a pump disposed along the second conduit between the third end and the fourth end.

In some embodiments, the device may further include a power source coupled to the pump. In some embodiments, the power source includes a transcutaneous energy transfer (TET) system. In some embodiments, the TET system may include an internal TET controller and telemetry system, an external TET controller and telemetry system, a primary coil coupled to the external TET controller and telemetry system, a secondary coil coupled to the internal TET controller and telemetry system, and a battery pack coupled to the external TET controller and telemetry system.

In some embodiments, the pump includes an axial flow pump, a peristaltic pump, or a combination thereof. In some embodiments, the pump is programmable to set a pumping rate, a flow type, or a combination thereof. In some embodiments, the flow type includes continuous flow, variable flow, pulsatile flow, or a combination thereof. In some embodiments, the pumping rate is programmable from 0.5-2.5 liters per minute.

According to at least one embodiment of the disclosure, a method of delivering blood from a lower pressure region to a higher pressure region may include flowing, through a first conduit disposed in the higher pressure region, a first portion of blood from the higher pressure region, in which a first end of the first conduit is coupled to the higher pressure region at a first location and a second end of the first conduit is coupled to the higher pressure region at a second location downstream of the first location. The method may also include diverting, through a second conduit, a second portion of blood from the lower pressure region, in which a third end of the second conduit is fluidly coupled to the lower pressure region and a fourth end of the second conduit is fluidly coupled to a narrow segment of the first conduit, the narrow segment disposed between the first end and the second end. The method may also include drawing the second portion of blood from the second conduit into the first conduit, at least in part, by pumping, with a pump, the second portion of blood along the second conduit. The method may further include providing the first portion of blood and the second portion of blood to the higher pressure region at the second end of the first conduit.

In some embodiments, the lower pressure region includes a left atrium of a heart and the higher pressure region includes an aorta.

In some embodiments, drawing the second portion of blood from the second conduit into the first conduit further includes generating, with the narrow segment, a Venturi effect.

In some embodiments, the method further includes programming the pump to set a pumping rate, a flow type, or a combination thereof.

In some embodiments, the method further includes powering the pump with a power source comprising a TET system.

### BRIEF DESCRIPTION OF THE DRAWINGS

FIG. 1 is an illustration of a device coupled to a heart and an aorta according to at least one embodiment of the present disclosure.

FIG. 2 is an illustration of a portion of the device illustrated in FIG. 1.

FIG. 3 is an illustration of a portion of the device illustrated in FIGS. 1 and 2.

FIG. 4 is a flow chart of a method according to at least one embodiment of the present disclosure.

FIG. 5 is a flow chart of a method according to at least one embodiment of the present disclosure.

FIG. 6 illustrates the simulated control and devices according to example embodiments of the present disclosure.

FIG. 7 shows plots of the blood flow rate overtime for the Control Model.

FIG. 8 shows plots of the blood flow rate over time for Model 0 according to an example embodiment of the disclosure.

FIG. 9 shows plots of the blood flow rate over time for Model 0 according to an example embodiment of the disclosure.

FIG. 10 shows plots of the blood flow rate over time for Model 1 according to an example embodiment of the disclosure.

FIG. 11 shows plots of the blood flow rate over time for Model 1 according to an example embodiment of the disclosure.

FIG. 12 shows plots of the blood flow rate over time for Model 2 according to an example embodiment of the disclosure.

FIG. 13 shows plots of the blood flow rate over time for Model 3 according to an example embodiment of the disclosure.

FIG. 14 is a chart comparing the performance of Model 0 to the Control Model.

FIG. 15 shows flow rates over time during one cardiac cycle for three cardiac outputs according to embodiments of the present disclosure.

FIG. 16 is a chart comparing the performance of Model 1 to the Control Model.

FIG. 17 shows flow rates over time during one cardiac cycle for three cardiac outputs according to embodiments of the present disclosure.

FIG. 18 is a plot of flow rates over time for one cardiac cycle in the descending aorta for a fixed cardiac output according to examples of the present disclosure.

FIG. 19 is a plot of flow rates over time for one cardiac cycle in the subclavian artery for a fixed cardiac output according to examples of the present disclosure.

FIG. 20 shows pressure contours of the control and the example devices in middle systole according to embodiments of the present disclosure.

FIG. 21 shows pressure contours of the control and the example devices in middle diastole according to embodiments of the present disclosure.

### DETAILED DESCRIPTION

The following description of certain embodiments is merely exemplary in nature and is in no way intended to limit the disclosure or its applications or uses. In the following detailed description of embodiments of the present devices, apparatuses, systems, and methods, reference is made to the accompanying drawings which form a part hereof, and which are shown by way of illustration specific embodiments in which the described devices, apparatuses, systems, and methods may be practiced. These embodiments are described in sufficient detail to enable those skilled in the art to practice presently disclosed devices, apparatuses, systems, and methods and it is to be understood that other embodiments may be utilized and that structural and logical changes may be made without departing from the spirit and scope of the present disclosure. Moreover, for the purpose of clarity, detailed descriptions of certain features, such as well-known anatomical structures and medical conditions, will not be discussed when they would be apparent to those with skill in the art so as not to obscure the descriptions of the present disclosure. The following detailed description is therefore not to be taken in a limiting sense, and the scope of the present devices, apparatuses, systems, and methods is defined only by the appended claims.

A healthy left ventricle must generate a significant level of pressure when it contracts (e.g., systole), and thereby ejects oxygenated blood, in order to generate sufficient flow of blood through the body. Pressure in the aorta, which receives the oxygenated blood from the left ventricle, is also high, particularly during ventricular systole. In contrast, the left atrium, which receives oxygenated blood from the lungs via the pulmonary veins and provides the blood to the left ventricle, has a lower pressure than the left ventricle and aorta during ventricular systole.

It may be desirable to provide a technique for delivering blood from a lower pressure region to a higher pressure region with reduced complexity and/or power requirements compared to existing techniques. Such a technique may allow more flexibility in selecting a power source and/or reducing the size and/or power of pump required. In some applications, this may reduce secondary procedures, such as surgical procedures, and/or reduce morbidities associated with failures of power sources and/or complex devices.

The Venturi effect is the reduction in pressure of a fluid flowing in a conduit as the fluid flows through a narrow segment of the conduit. The pressure of the fluid in the conduit upstream from the narrow segment is greater than the pressure of the fluid in the narrow segment. This is due, at least in part, to an increase in velocity of the fluid as it passes through the narrow segment. The Venturi effect may be utilized to deliver blood from a lower pressure region to a higher pressure region without the use of a pump or other powered device in some applications.

According to embodiments of the present disclosure, a conduit with a narrow segment may be disposed in a higher pressure region. The conduit may be disposed such that blood within the high pressure region flows through the conduit. Blood may be delivered from a lower pressure region to the narrow segment of the conduit. The narrow segment may provide a pressure drop (e.g., due to the Venturi effect) that allows the blood to flow from the lower pressure region to the higher pressure region. In some embodiments, a pump may be located along a second conduit to assist the flow of blood from the lower pressure region to the higher pressure region. In some applications, devices and methods disclosed herein may utilize the Venturi effect, at least in part, to deliver oxygenated blood from the left atrium or pulmonary vein to the aorta. In other words, the Venturi effect may assist the pump to deliver the blood from the lower pressure region to the higher pressure region. In some applications this may reduce the power and/or size requirements of the pump.

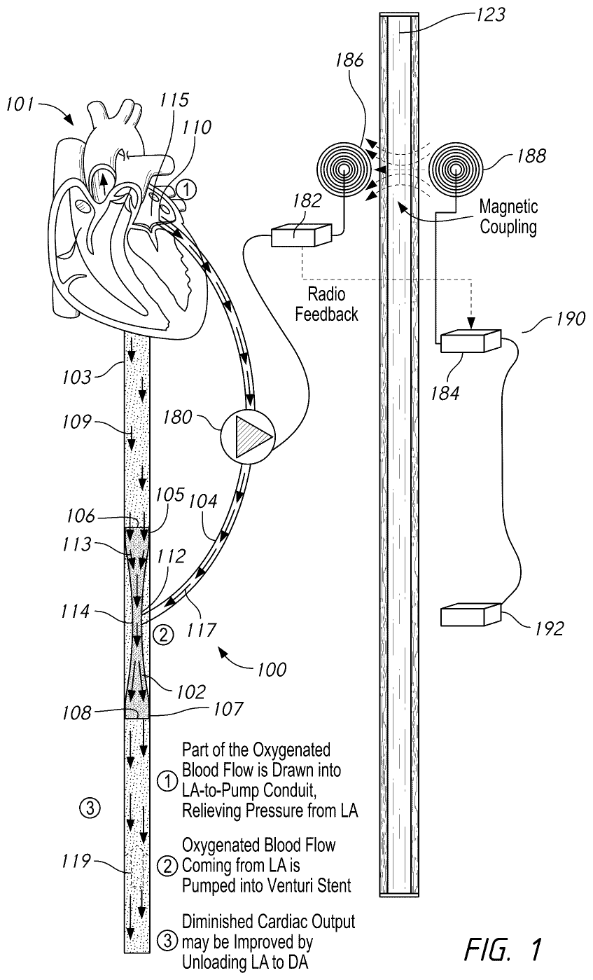

FIG. 1 is an illustration of a device coupled to a heart and an aorta according to at least one embodiment of the present disclosure. Device 100 may be coupled to a higher pressure region and a lower pressure region. In the example shown in FIG. 1, device 100 is coupled to a left atrium 115 of a heart 101 and an aorta 103. In this example, the left atrium 115 may be a lower pressure region and the aorta 103 may be a higher pressure region. However, the lower and higher pressure regions may be different in other examples. For example, the lower pressure region may be a pulmonary vein in other examples and the higher pressure region may be a descending aorta.

The device 100 may include a first conduit 102 and a second conduit 104. The first conduit 102 may be disposed within the aorta 103. A long axis of the first conduit 102 may be parallel to and/or aligned with a long axis of the aorta 103. The first conduit 102 may have a first end 106 located within the aorta 103 at a first site 105 and a second end 108 located within aorta 103 at a second site 107. The first site 105 may be upstream with respect to blood flow through the aorta (indicated by arrow 109) and/or proximate the heart 101 relative to second site 107, which may be downstream and/or distal to the heart 101 relative to first site 105. In some embodiments, the first conduit 102 may be mechanically coupled to the aorta 103 (e.g., sutures, staples). In some embodiments, the first conduit 102 may be coupled to the aorta 103 by friction and/or compression (e.g., the first conduit 102 may have an expandable stent structure). Some or all of the blood flowing through the aorta 103 may flow through first conduit 102 as indicated by arrow 113.

The second conduit 104 may have a first end 110 fluidly coupled to the left atrium 115 and a second end 112 coupled to the first conduit 102 (the first end 110 and second end 112 of the second conduit 104 may also be referred to as third and fourth ends, respectively). The second conduit 104 may divert at least a portion of the blood in the left atrium 115 to the first conduit 102. The second end 112 of the second conduit 104 may be coupled to a narrow segment 114 of the first conduit 102 disposed between the first end 106 and the second end 108 of the first conduit 102. The narrow segment 114 may have a diameter that is narrower than a diameter of the first conduit 102 at the first end 106 and the second end 108.

In some embodiments, such as the one shown in FIG. 1, device 100 may include a pump 180 disposed at a location along the second conduit 102 between the first end 110 and second end 112. Although shown approximately midway along the second conduit 102 in FIG. 1, in other embodiments, the pump 180 may be located closer to the first end 110 or second end 112. The pump 180 may pump blood through the second conduit 104 from the left atrium 115 to the first conduit 102. In some embodiments, the pump 180 may be implemented by an axial flow pump. In other embodiments, other pump types may be used, such as a peristaltic pump. In some embodiments, the pump 180 may be programmable. For example, the pump 180 may be programmable to set a speed (e.g., pumping rate) and/or a flow type such as variable, pulsatile, and/or continuous flow. In some examples, the pump may have a pumping rate between 0.5-2.5 liters per minute.

The pump 180 may be powered by a power source. In some embodiments, the power source may include a transcutaneous energy transfer (TET) system 190. In some embodiments, the TET system 190 may include an internal TET controller and telemetry system 182, an external TET controller and telemetry system 184, a secondary coil 186, a primary coil 188, and a battery pack 192. The internal TET controller and telemetry system 182 and secondary coil 186 may be implanted under the skin 123 of a subject. For example, just below the clavicle, similar to where pacemaker batteries are typically implanted. Energy from the battery pack 192 may be transmitted from the primary coil 188 through the skin 123 to the secondary coil 186 to the pump 180. The transmission of the energy may be controlled by the internal TET controller and telemetry system 182 and/or the external TET controller and telemetry system 184. In some embodiments, energy may be transmitted between the primary coil 188 and secondary coil 186 via magnetic coupling. Details of suitable TET systems that may be used to implement the TET system 190 may be found at: Dissanayake, T. & Budgett, D. & Hu, Aiguo & Malpas, Simon & Bennet, L. (2009). Transcutaneous Energy Transfer System for Powering Implantable Biomedical Devices, Au SLC, McCormick D. Lever N, Budgett D. Thermal evaluation of a hermetic transcutaneous energy transfer system to power mechanical circulatory support devices in destination therapy. Artif Organs. 2020 September; 44(9):955-967, Energy Transmission and Power Sources for Mechanical Circulatory Support Devices to Achieve Total Implantability, Wang, Jake X. et al. The Annals of Thoracic Surgery, Volume 97, Issue 4, 1467-1474, U.S. Patent Application Publication 2021/0351628, and the FREE-D Wireless Energy Transfer System described by the Bonde Artificial Heart Lab at the Yale School of Medicine (https://medicine.yale.edu/lab/bonde/research/free-d/), all of which are incorporated herein by reference for any purpose.

In some implementations, the Venturi effect produced in the narrow segment 114 may assist the pump 180 in delivering blood from the left atrium 115 to the aorta 103, such as by reducing the amount of power needed to deliver the blood. The power requirements of the pump 180 may be reduced compared to a device similar to device 100 but in which a Venturi effect is not produced. The pump 180 may operate at a reduced power output compared to a device similar to device 100 but that does not include a narrow segment 114. Alternatively or additionally, the size requirements of the pump 180 may be reduced compared to a similar device in which a Venturi effect is not produced. Alternatively or additionally, the demands on, or requirements of, any component of the TET system 190 may be reduced compared to a device similar to device 100 but in which a Venturi effect is not produced.

Returning to the first conduit 102 and the second conduit 104, based, at least in part, on the Venturi effect, the fluid pressure within the narrow segment 114 may be less than the fluid pressure in the aorta 103 and other portions of the first conduit 102. The pressure in the narrow segment 114 may be low enough such that blood is drawn from the second conduit 104 into the first conduit 102 as indicated by arrow 117. In some embodiments, the pressure in the narrow segment 114 may be lower than the pressure in the second conduit 104. In some embodiments, the Venturi effect may assist the pump 180 in transferring blood from the left atrium 115 to the aorta 103. In some embodiments, the Venturi effect may reduce the power and/or size requirements of the pump 180 to achieve a desired flow rate.

The blood from the left atrium 115 diverted through the second conduit 102 may be combined with the blood of the aorta 103. Thus, the blood diverted from the left atrium 115 may bypass the left ventricle of the heart 101. In some applications, bypassing the left ventricle with device 100 may at least partially compensate for poor ejection fraction of the left ventricle.

During ventricular diastole, blood in the aorta 103 may temporarily reverse flow as indicated by arrow 119. If the reverse blood flow in the aorta 103 were to push blood from the first conduit 102 into the second conduit 104 and into the left atrium 115, this could cause damage to the heart 101. Accordingly, back flow of blood into the left atrium 115 is undesirable. In some embodiments, the Venturi effect may obviate the need for valves or other components for preventing backflow of blood from the aorta 103 and/or first conduit 102 into the second conduit 104 and/or left atrium 115. During ventricular diastole, pressure in the aorta 103 may decrease and the pressure in the narrow segment 114 may be lower during ventricular diastole than during ventricular systole. Due, at least in part, to the Venturi effect in the narrow segment 114, the pressure in the second conduit 104 may be equal to or greater than the pressure in the narrow segment 114 during ventricular diastole. The higher pressure in the second conduit 104 compared to the narrow segment 114 may prevent backflow of blood into the left atrium 115 in some embodiments. In some embodiments, the pump 180, alternatively or additionally, may inhibit backflow of blood into the left atrium 115.

In some embodiments, the first conduit 102 and second conduit 104 may be of unitary construction. That is, device 100 may be formed as a single piece or unit. In some embodiments, the device 100 may be free of seams or joints between the first conduit 102 and the second conduit 104. In some embodiments, the first conduit 102 and the second conduit 104 may be separate components that are coupled during implantation. For example, the first conduit 102 may be delivered to the aorta 103 via a catheter (not shown). In some embodiments, this procedure may be similar to placing a stent. The second conduit 104 may be implanted and coupled to the left atrium 115 and the first conduit 102 via a thoracoscopy. In some embodiments, the device 100 may be delivered via a catheter, even when of unitary construction. In some embodiments, a thoracoscopy may be used to “retrieve” the first end 110 of the second conduit 104 from the aorta 103 and couple it to the left atrium 115. In some embodiments, device 100 may be implanted entirely by thoracoscopy. In embodiments including pump 180, the pump 180 and/or power source (e.g., TET system 190) may be implanted via thoracoscopy. In some embodiments, a percutaneous procedure and/or thoracotomy may be used in addition to or instead of a thoracoscopy to implant some or all of the components.

While the embodiment shown in FIG. 1 illustrates the first conduit 102 within the aorta 103, in some embodiments, the aorta 103 may be cut and the resulting ends coupled to the ends of the device 100 (e.g., via clamps, sutures, or other attachment means). For example, the first site 105 may be coupled to the first end 106 and the second site 107 may be coupled to second end 108. In some embodiments, a portion of the aorta 103 may be removed. In some embodiments, the portion removed may be approximately equal to a length of the first conduit 102.

In some embodiments, the device 100 may be constructed of a biocompatible material. The biocompatible material may be polytetrafluoroethylene (e.g., Teflon). In some embodiments, the device 100 may include one or more coatings on the interior and/or exterior surfaces of the conduits 102, 104, such as anti-coagulant and/or anti-fouling coatings.

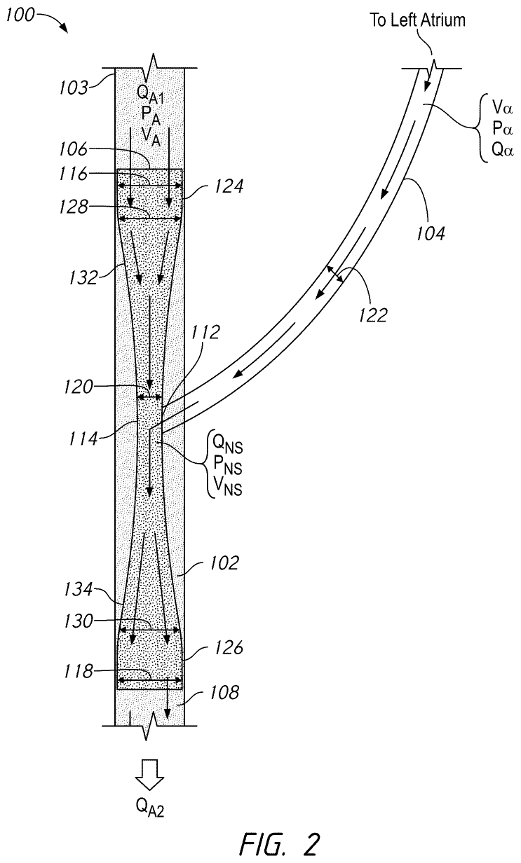

FIG. 2 is an illustration of a portion of the device illustrated in FIG. 1. FIG. 2 is a magnified view of the device 100 near the narrow segment 114. While a portion of the aorta 103 is shown for context, the heart 101 and the first end 110 of the second conduit 104 are not shown in FIG. 2. In some embodiments, the first conduit 102 may have a first diameter 116 at the first end 106 and a second diameter 118 at the second end 108. In some embodiments, the diameters 116 and 118 may be the same (which, as used herein, may mean equal, substantially equal). In some embodiments, the diameter 116 and/or diameter 118 may be the same as a diameter of the aorta at the respective location. In some embodiments, a segment 124 of the first conduit 102 between the first end 106 and the narrow segment 114 may have the same diameter as diameter 116 until a transition segment 132. Thus, diameter 128 may be the same as diameter 116. Similarly, a segment 126 of the first conduit 102 between the second end 108 and the narrow segment 114 may have the same diameter as diameter 118 until a transition segment 134. Diameter 130 may be the same as diameter 118. As used herein, diameter refers to an inner diameter of the conduits 102, 104. In some embodiments, diameter 116, 118, 128, and/or 130 may be the same as a diameter of the aorta 103. In some embodiments, a length of segment 124 and a length of segment 126 may be the same. In some embodiments, the narrow segment 114 may be disposed equidistant between the first end 106 and the second end 108. However, in other embodiments, segments 124 and 126 may have different lengths. In some embodiments, segment 114 may be disposed closer to the first end 106 than the second end 108, or closer to the second end 108 than the first end 106.

In some embodiments, the narrow segment 114 may have a diameter 120 that is less than at least one of diameter 116 and diameter 118. In some embodiments, the diameter 120 may be 40% to 60% (inclusive) of diameter 116 and/or diameter 118. In some embodiments, the narrow segment 114 may have the same diameter for the length of the narrow segment 114. The second conduit 104 may have a diameter 122. In some embodiments, the second conduit 104 may have the same diameter from the first end 110 (shown in FIG. 1) to the second end 112. In some embodiments, the diameter 122 may be to the same as the diameter 120 of the narrow segment 114.

The difference between diameter 120 of the narrow segment 114 and the other portions of the first conduit 102 may generate a Venturi effect when fluid, such as blood, flows through the first conduit 102. The Venturi effect may be described by:

$${P}_{A} - {P}_{NS} = ( {ρ}_{B} / 2 ) ( {V}_{NS}^{2} - {V}_{A}^{2} ) \qquad Equation 1$$

Where P_{A} is the pressure in the aorta, P_{NS} is the pressure in the narrow segment 114, ρ_{B} is the density of blood, V_{NS} is the velocity of blood in the narrow segment 114, and V_{A} is the velocity of blood in the aorta 103. As provided by Equation 1, as velocity in the narrow segment 114 increases relative to the velocity in the aorta 103, the pressure in the narrow segment 114 decreases relative to the pressure in the aorta 103. Velocity of a fluid through a cylindrical (or substantially cylindrical) conduit, such as conduit 102, conduit 104, and/or aorta 103 is provided by:

$$V = \frac{Q}{A} \qquad Equation 2$$

Where Q is flow and A is the area of a cross section of the conduit. The area may be provided by:

$$A = {π ( d / 2 )}^{2} \qquad Equation 3$$

Where d is the diameter of the conduit. As shown by Equations 2-3, the velocity is inversely proportional to the diameter of the conduit. Thus, as the diameter of the conduit decreases, the velocity increases, which in turn leads to a decrease in pressure. Based on the underlying physics of the Venturi effect and generally accepted principles of physiology, the following assumptions may be made:

$${V}_{NS} > {V}_{A} \qquad Equation 4$$

$${P}_{NS} < {P}_{A} \qquad Equation 5$$

$${Q}_{A 2} = {Q}_{A 1} + {Q}_{α} \qquad Equation 6$$

$${P}_{α} = {P}_{LA} + {ρ}_{B} gh \qquad Equation 7$$

$${V}_{NS} {A}_{NS} = {V}_{A} {A}_{A} \qquad Equation 8$$

Where V_{NS} is the blood velocity in the narrow segment 114, V_{A} is the blood velocity in the aorta, and V_{α} is a velocity of blood in the second conduit 104, Q_{A1} is the blood flow through the aorta 103 upstream from the first end 106 of the first conduit 102, Q_{α} is the blood flow through the second conduit 104, Q_{NS} is the blood flow through the narrow segment 114, and Q_{A2} is the blood flow through the aorta 103 downstream from the second end 108 of the first conduit 102. Further, P_{α} is the pressure in the second conduit 104, P_{LA} is the pressure in the left atrium 115, g is the acceleration due to gravity, h is the distance between the site where the first end 110 of the second conduit 104 is coupled to the left ventricle 115 and the site where the second end 112 of the second conduit 104 is coupled to the narrow segment 114, A_{NS} is an area of a cross section of the narrow segment 114, and A_{A} is an area of a cross section of the aorta 103.

In some applications, the goal may be to maximize Q_{α} and the diameters of the various segments 114, 124, 126 of first conduit 102 and/or the diameter 122 of the second conduit 104 may be adjusted relative to one another accordingly based, at least in part, on Equations 1-8 above. Returning to Equation 8, V_{NS} may be provided by:

$${V}_{NS} = {V}_{A} \frac{{A}_{A}}{{A}_{NS}} = {{V}_{A} ( \frac{{r}_{A}}{{r}_{NS}} )}^{2} \qquad Equation 9$$

Where r_{A} is the radius (e.g., half of the diameter) of the aorta 103 and r_{NS} is the radius (e.g., half of the diameter 120) of the narrow segment 114. In some embodiments, the diameter of the aorta 103 may be the same as the diameter 116, 128, 130, and/or 118 of the first conduit 102. Plugging Equation 13 into Equation 11, provides:

$$\frac{2 ( {P}_{A} - {P}_{NS} )}{{ρ}_{B}} = {V}_{A}^{2} [ {( \frac{{r}_{A}}{{r}_{NS}} )}^{4} - 1 ] \qquad Equation 10$$

In embodiments when a pump, such as pump 180, is not included, to allow blood to flow from the lower pressure region (e.g., left atrium) to the higher pressure region (e.g., aorta), the pressure in the narrow segment 114 must be less than the pressure P_{α} of the second conduit 104:

$${P}_{NS} < {P}_{LA} + {ρ}_{B} gh \qquad Equation 11$$

By plugging Equation 11 into Equation 10, it can be derived:

$$1 + \frac{2 ( {P}_{A} - {P}_{LA} - {ρ}_{B} gh )}{{ρ}_{B} {V}_{A}^{2}} = {( \frac{{r}_{A}}{{r}_{NS}} )}^{4} \qquad Equation 12$$

To simplify:

$$m = \frac{2 ( {P}_{A} - {P}_{LA} - {ρ}_{B} gh )}{{ρ}_{B} {V}_{A}^{2}} \qquad Equation 13$$

Where m represents the portion of the pressures of the device 100 and surrounding anatomy that does not depend on diameters of the conduits or anatomy. In some applications, m may represent pressures that cannot be controlled by adjusting various dimensions of the device 100 and/or surrounding anatomy. The simplified version of Equation 13 is given by:

$$1 + m = {( \frac{{r}_{A}}{{r}_{NS}} )}^{4} \qquad Equation 14$$

Thus, the ratio of the radius (e.g., half the diameter 120) of the narrow segment 114 and the radius of the aorta 103 that provides for a sufficient Venturi effect is provided by:

$$\frac{{r}_{NS}}{{r}_{A}} < {( \frac{1}{1 + m} )}^{1 / 4} \qquad Equation 15$$

Thus, Equation 15 may be used to determine a suitable radius, and thus a suitable diameter 120, for the narrow segment 114 when the diameter 116, 128, 130, and/or 118 of the first conduit 102 is to the same as the diameter of the aorta 103.

However, in embodiments where a pump, such as pump 180, is included to assist drawing blood from the left atrium 115 to the aorta 103, the ratio of the radius of the narrow segment 114 and the radius of the aorta 103 that provides for a sufficient Venturi effect may be greater. That is, in some embodiments, the diameter 120 of the narrow segment 114 may not need to be as narrow compared to the diameter of the aorta 103 when a pump is used.

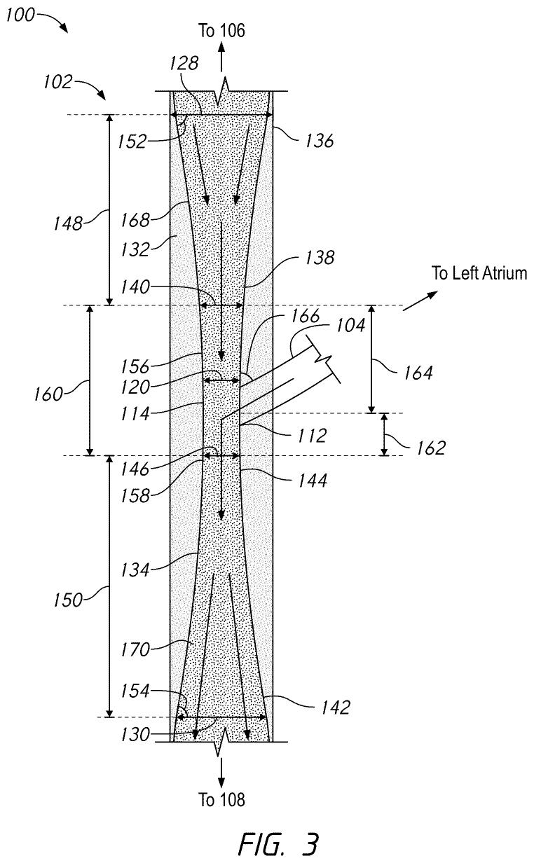

FIG. 3 is an illustration of a portion of the device illustrated in FIGS. 1 and 2. FIG. 3 is a magnified view of the device 100 near the narrow segment 114. The first end 106 and second end 108 of the first conduit 102 and the first end 110 of the second conduit 104 are not shown in FIG. 3. In some embodiments, the first conduit 102 may gradually change diameters from diameters 128 and 130 to the diameter 120 of the narrow segment 114 in transition segments 132 and 134. The transition segments 132 and 134 may have truncated conical structures with diameters that vary linearly over distance in some embodiments, such as the one shown in FIG. 3. However, in other embodiments, the transition segments 132 and 134, independently of each other, may have different structures with diameters that change differently with distance (e.g., hyperbolic, polynomial).

In the example shown in FIG. 3, transition segment 132 may have a first end 136 proximate the first end 106 of the first conduit 102 (shown in FIGS. 1-2) that has a diameter 128 and a second end 138 proximate the narrow segment 114 that has a diameter 140 (the first end 136 and the second end 138 of the transition segment 132 may also be referred to as fifth and sixth ends, respectively). In some embodiments, diameter 140 may be the same as diameter 120 of the narrow segment 114. The transition segment 132 may have a length 148. The length 148 and difference between diameters 128 and 140 may provide for an angle 152 (e.g., convergent angle) between an interior wall 168 of the transition segment 132 and a plane perpendicular to the flow of fluid through the conduit 102. As the difference between diameters 128 and 140 increases, the angle 152 decreases when the length 148 is held constant. As the length 148 is increased, the angle 152 increases when the difference between diameters 128 and 140 are held constant.

Transition segment 134 may have a first end 142 proximate the second end 108 of the first conduit 102 (shown in FIGS. 1-2) that has a diameter 130 and a second end 144 proximate the narrow segment 114 that has a diameter 146 (the first 142 and second 144 ends of the transition segment 134 may also be referred to as seventh and eighth ends, respectively). In some embodiments, diameter 146 may be the same as diameter 120 of the narrow segment 114. The transition segment 134 may have a length 150. The length 150 and difference between diameters 130 and 146 may provide for an angle 154 (e.g., divergent angle) between an interior wall 170 of the transition segment 134 and a plane perpendicular to the flow of fluid through the conduit 102. As the difference between diameters 130 and 146 increases, the angle 154 decreases when the length 150 is held constant. As the length 150 is increased, the angle 154 increases when the difference between diameters 130 and 146 are held constant.

In some embodiments, the dimensions of the transition segments 132 and 134 may be the same. For example, diameters 128 and 130 may be the same, diameters 140 and 146 may be the same, and lengths 148 and 150 may be the same. However, in some embodiments, the transition segments 132 and 134 may have one or more dimensions that differ. For example, diameters 128 and 130 may be different. In another example, lengths 148 and 150 may be different.

In some embodiments, the narrow segment 114 may have a first end 156 proximate the first transition segment 132 (which is proximate the first end 106 of the first conduit 102) and a second end 158 proximate the second transition segment 134 (which is proximate the second end 108 of the first conduit 102) and have a length 160 (the first 156 and second 158 ends of the narrow segment 114 may also be referred to as ninth and tenth ends, respectively). The length 160 may also be referred to as a “landing zone.” In some embodiments, the second end 112 of the second conduit 104 may be coupled to the narrow segment 114 at a location equidistant from the first end 156 and the second end 158. However, in other embodiments, the second end 112 of the second conduit 104 may be coupled to the narrow segment 114 at a location closer to the first end 156 or closer to the second end 158 of the narrow segment 114. In the example shown in FIG. 3, the second end 112 of the second conduit 104 is coupled to the narrow segment 114 at a location closer to the second end 158. Thus, a length 164 between the first end 156 and a center of the second end 112 of the second conduit 104 is greater than a length 162 between the second end 158 and the center of the second end 112 of the second conduit 104.

In some embodiments, the second end 112 of the second conduit 104 may be coupled to the narrow segment 114 at an angle 166. In some embodiments, such as the one shown in FIG. 3, the angle 166 is less than 90 degrees.

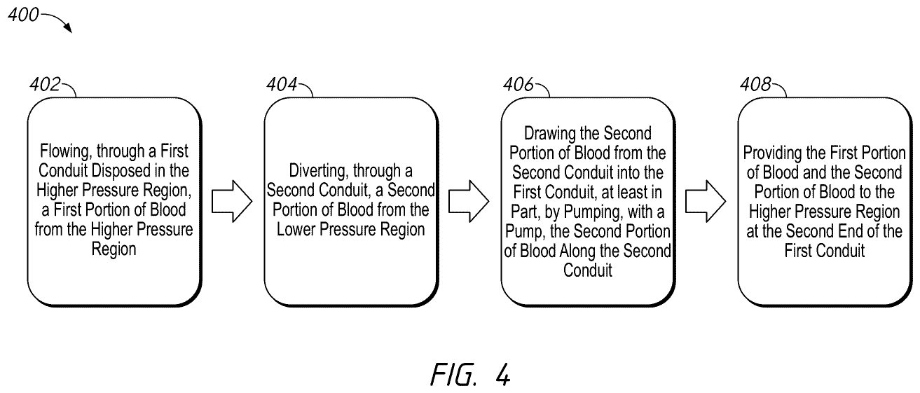

FIG. 4 is a flow chart of a method according to at least one embodiment of the present disclosure. Method 400 may be a method for delivering blood from a lower pressure region to a higher pressure region. In some embodiments, the method 400 may be performed by a device, such as device 100 in FIGS. 1-3.

At block 402, “flowing, through a first conduit disposed in the higher pressure region, a first portion of blood from the higher pressure region” may be performed. In some embodiments, the first conduit may be first conduit 102. In some embodiments, the higher pressure region may be an aorta, such as aorta 103. A first end of the first conduit may be coupled to the higher pressure region at a first location and a second end of the first conduit may be coupled to the higher pressure region at a second location downstream of the first location in some embodiments.

At block 404, “diverting, through a second conduit, a second portion of blood from the lower pressure region” may be performed. In some embodiments, the second conduit may be second conduit 104. In some embodiments, the lower pressure region may be a left atrium, such as left atrium 115. In some embodiments, a third end of the second conduit may be fluidly coupled to the lower pressure region and a fourth end of the second conduit may be fluidly coupled to a narrow segment of the first conduit, the narrow segment disposed between the first end and the second end.

At block 406, “drawing the second portion of blood from the second conduit into the first conduit, at least in part, by pumping, with a pump, the second portion of blood along the second conduit” may be performed. In some embodiments, the pump may be pump 180. In some embodiments, drawing the second portion of blood from the second conduit into the first conduit may further include generating, with the narrow segment, a Venturi effect.

At block 408, “providing the first portion of blood and the second portion of blood to the higher pressure region at the second end of the first conduit” may be performed.

Optionally, method 400 may further include programming the pump to set a pumping rate, a flow type, or a combination thereof. Optionally method 400 may further include powering the pump with a power source comprising a transcutaneous energy transfer (TET) system, such as TET system 190.

In some embodiments, the devices disclosed herein may be implanted in a subject without any cables or components protruding through the skin. In some embodiments, the devices disclosed herein may at least partially bypass a weakened and/or atrophied left ventricle. In some embodiments, the devices disclosed herein may restore adequate cardiac output. In some embodiments, the devices disclosed herein may alleviate elevated left atrial pressure (LAP), diminished left ventricular compliance, diminished diastolic function, and diminished cardiac output as well as pulmonary congestion, and exercise intolerance. In some embodiments, the devices disclosed herein may be implanted without open-chest surgery. Thus, the surgery for the devices disclosed herein may be less invasive than for other devices or techniques in some embodiments.

In some embodiments, the constant flow rates provided by the example devices in Models 0-3 (see Examples 1 and 2), including the flow rate through the second conduit, may be provided, at least in part, by a pump, such as pump 180.

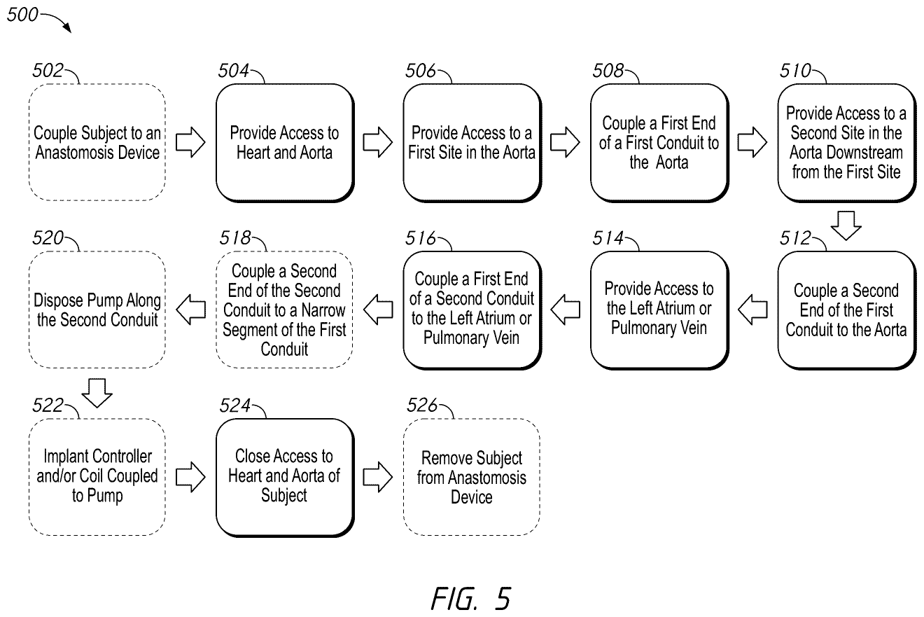

FIG. 5 is a flow chart of a method according to at least one embodiment of the present disclosure. Method 500 may be a method for installing (e.g., implanting) a device to deliver blood from a lower pressure region to a higher pressure region, such as device 100 in FIGS. 1-3. The device may be implanted in a subject, such as a person suffering from heart failure. In some applications, method 500 may be performed, at least in part, by a cardiac surgeon and/or other clinician. Thus, detailed explanations of procedures and surgical techniques within the skill of such clinicians (e.g., anesthesia, operation of a cardiac bypass machine) are not provided.

Optionally, as indicated at block 502, a subject may be coupled to an anastomosis device. Any suitable device and/or technique, such as a conduit anastomosis, may be used. In some implementations, the subject is coupled to a cardiac bypass machine. However, in some embodiments, the subject may not be coupled to a cardiac bypass machine and other techniques and/or devices may be used. Access to a heart (e.g., 101) and aorta (e.g., 103) of the subject may be provided as indicated by block 504. In an example, an incision may be made to access the heart and/or aorta. Any appropriate technique for making the incision may be used. In some embodiments, multiple incisions may be made. The location and/or number of incisions may be based, at least in part, on the chosen surgical method (e.g., thoracoscopy, laparoscopic, open chest). For example, when a catheter is used to implant at least a portion of the device (e.g., the first conduit 102), the incision may be made proximate the femoral vein or other suitable vein for introducing the catheter.

As indicated at block 506, access to a first site (e.g., 105) in the aorta may be provided and a first end (e.g., 106) of a first conduit (e.g., 102) may be coupled to the aorta at the first site to fluidly couple the first conduit to the aorta as indicated by block 508. Access to a second site (e.g., 107) in the aorta may be provided downstream from the first site and a second end (e.g., 108) of the first conduit may be coupled to the aorta at the second site as indicated by blocks 510 and 512. In some embodiments, the first and second access sites in the aorta may be based, at least in part, on a condition of the patient (e.g., partial ejection fraction), location and/or size of the aorta, and/or the relative locations of the left atrium and aorta to one another. In some embodiments, the first and second sites may be selected based, at least in part, on a size of the device, a diameter of the first conduit, and/or a length of the first conduit. Coupling between the ends of the first conduit and the sites of the aorta may be achieved by any suitable technique, such as by friction and/or compression (e.g., the first conduit may include an expandable stent structure), or by mechanical techniques (e.g., sutures, staples).

As shown in blocks 514 and 516, access may be provided to the left atrium (e.g., 115) or a pulmonary vein and a first end (e.g., 110) of a second conduit (e.g., 104) may be coupled at the access site to fluidly couple the second conduit to the left atrium or pulmonary vein. In some embodiments, the location of the access site in the left atrium or pulmonary vein may be based, at least in part, on the amount of blood to be diverted, a diameter of the second conduit, a length of the second conduit, the location where the first conduit is installed, and/or anatomical limitations (e.g., a suitable distance away from the pulmonary vein and/or heart valves, a location where the pulmonary vein is of suitable thickness). In some examples, an incision may be made to provide access to the aorta and left atrium. A scalpel, cauterizing scalpel, or other suitable tool may be used to cut openings into the tissue of the aorta, pulmonary vein, and/or left atrium to fluidly couple these regions to the device. The second conduit may be coupled to the left atrium or pulmonary vein by any suitable technique. For example, sutures, staples, and/or clamps may be used. Optionally, in embodiments when the first and second conduits are not formed as an integral unit, as indicated by block 518, a second end (e.g., 112) of the second conduit may be coupled to a site along a narrow segment (e.g., 114) of the first conduit via an incision in the aorta. In some embodiments, when the first and second conduits are formed as an integral unit (or are already coupled together), an incision may be made in the aorta to retrieve the first end of the second conduit prior to coupling the first end to the left atrium or pulmonary vein.

Optionally, in embodiments that include a pump (e.g., pump 180), the pump may be disposed along the second conduit as indicated by block 520. However, in some embodiments, the pump may already be disposed along the second conduit. In some embodiments, a controller (e.g., TET controller and telemetry system 182) and a coil (e.g., secondary coil 186) coupled to the pump 180 may be provided as indicated by block 522. The implanted controller and coil may be configured to interact with an external controller (e.g., external TET controller and telemetry system 184) and coil (e.g., primary coil 188), which may be coupled to a power source (e.g., battery pack 192). The controllers, coils, and power source may comprise a transcutaneous energy transfer (TET) system (e.g., TET system 190), which may power and/or control the pump.

The order of the blocks 506-522 are provided merely as an example, and the blocks 506-522 may be arranged in different orders. For example, blocks 510 and 512 may be performed prior to blocks 506 and 508. In some embodiments, blocks 514 and 516 may be performed before blocks 510 and 512 and/or before blocks 506 and 508. In some embodiments, block 518 may be performed prior to blocks 506, 508, 510, 512, 514, and/or 516. In some embodiments, all three incisions indicated by blocks 506, 510, and 514 may be performed prior to all of the coupling indicated by blocks 508, 512, and 516.

Although not shown in FIG. 5, in some embodiments, method 500 may optionally include trimming the lengths of the first and/or second conduit prior to coupling. That is, the clinician may finalize the length of the first and/or second conduit during implantation. For example, it may not be possible to obtain precise anatomical measurements of the subject prior to implantation in some applications. Thus, the clinician may not know until the performance of method 500 the precise lengths of the conduits that are required.

After blocks 504-516 (and optionally 502, 518, 520, and/or 522) have been performed, the access to the heart and aorta of the subject may be closed as indicated by block 524. Any suitable technique for closing the access may be used (e.g., sutures, staples, glue). As indicated by block 526, optionally, the subject may be removed from the anastomosis device if block 502 was performed. For example, the subject may be removed from a bypass machine if a bypass machine is used. As noted, in some applications, a bypass machine may not be used. For example, a proximal clampless anastomotic device, or similar device, and/or catheters may be used to perform block 506, 508, 510, 512, 514, and/or 516 and may obviate the need for a bypass machine or other similar device, to perform method 500. An example of a proximal clampless anastomotic device is the PAS-Port System by Cardica, Inc. (Redwood City, California). However, performing method 500 without a bypass machine is not limited to this particular device.

Prior to performing method 500, blood may flow through the lungs to the pulmonary veins back to the heart to fill the left atrium. The left atrium may pump blood into the left ventricle. The left ventricle may then eject the received blood into the aorta. The blood may flow through the aorta to be distributed to smaller vessels throughout the body. After method 500 is performed, some of the blood arriving from the pulmonary veins to the left atrium flows through the second conduit to the narrow segment. The remaining blood in the left atrium may be delivered to the left ventricle. At least some of the blood in the left ventricle may then be pumped into the aorta. After method 500 is performed, blood flowing through the aorta may flow through the first conduit.

As the blood flows through the narrow segment, the velocity of the blood may increase while the fluid pressure of the blood decreases due to the Venturi effect. The decrease in pressure of the blood in the narrow segment may draw the portion of blood that flowed through the second conduit into the narrow segment. Thus, the blood from the aorta may be combined with the blood diverted from the left atrium where the second end of the second conduit is coupled to the narrow segment. The combined blood flows from the first end of the first conduit and the second conduit may flow through the first conduit from the narrow segment to the second end and flow into the aorta for delivery to the rest of the body.

In embodiments, the drawing of blood from the left atrium into the aorta due the Venturi effect may be supplemented by the use of a pump (e.g., pump 180). The pump may increase the volume of blood that may be drawn from the left atrium into the aorta compared to when only the Venturi effect is used.

The blood flowing from the second end of the first conduit may be combined with the blood flowing through the aorta. The blood provided to the aorta at the second end of the first conduit includes the blood pumped by the left ventricle into the aorta and the blood diverted from the left atrium through the device. In some applications, the amount of blood in the aorta available for distribution throughout the body may be greater than the amount of blood available for distribution prior to performance of method 500.

### EXAMPLES

Various example embodiments of the present disclosure are provided herein. In particular, various example embodiments of devices, such as device 100, are provided herein. The examples are provide merely for explanatory purposes and the disclosure is not limited to the specific examples provided herein.

### Example 1—Model Generation

For the purpose of the present examples, an aortic model was created based on computed tomography (CT) scans of a 23-year-old male (Model #94) from the Vascular Model Repository (https://www.vascularmodel.com/). SimVascular was then used for image segmentation, geometry reconstruction, mesh generation, and a patient-specific flow simulation and analysis. The example devices were placed in the descending aorta of the simulation. As a control, simulations were also performed on the model with no device. While the CT scans were used for generating simulations for these examples, in some embodiments, acquiring of CT scans from patients and analysis of the resulting images with software, such as SimVascular, may be used to design custom devices for patients. For example, the lengths of the first and second conduits, initial widths of the first and second ends of the first conduit, and diameter of the narrow segment may be determined based on the analyzed CT scans.

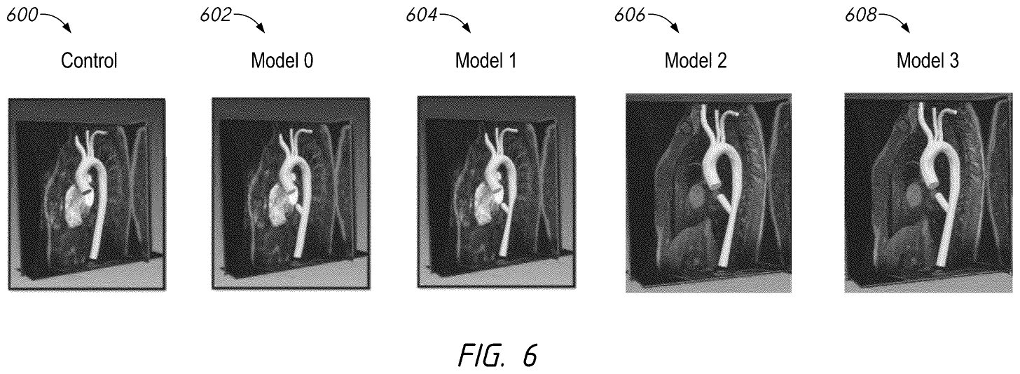

FIG. 6 shows images of the simulated control and devices according to example embodiments of the present disclosure. Image 600 shows a simulated aorta with no device (“Control Model”). Image 602 shows a simulated aorta including an example device with a constant diameter (Model 0). Image 604 shows a simulated aorta including a narrow segment (Model 1). In particular, the narrow segment shown in image 604 is the narrowest of the example devices shown in FIG. 6. Images 606 and 608 also show simulated aortas including example devices with narrow segments (Models 2 and 3, respectively). More details on each of the models is provided below.

### Model 0

For Model 0, the first conduit (e.g., first conduit 102) of the device was omitted. The second conduit (e.g., second conduit 104), coupled between the left atrium and the aorta, had a uniform diameter of 12 mm.

### Model 1

For Model 1, the first conduit had a first transition segment (e.g., transition segment 132) 3.43 cm long and a second transition segment (e.g., transition segment 134) that was 6.87 cm long. The narrow segment was 20 mm in length. The total length for the first conduit was 12.29 cm. The second conduit (e.g., second conduit 104) coupled between the left atrium and the first conduit had a maximum diameter of 12 mm with a 7.5 mm “nozzle” where it coupled to the narrow segment. A ratio between the cross-sectional area (also referred to herein simply as “area”) of the aorta and the area of the narrow segment was 35%. That is, the narrow segment had an area that was 35% of the area of the aorta. The cross-sectional area is provided by:

$$A = π {r}^{2} \qquad Equation 16$$

Where A is the cross-sectional area and r is the radius.

For Model 1, the diameter of the aorta was 1.46 cm, and the radius of the narrow segment was approximately 0.25 cm.

### Model 2

For Model 2, the first conduit had a first transition segment (e.g., transition segment 132) 3.43 cm long and a second transition segment (e.g., transition segment 134) that was 6.87 cm long. The narrow segment was 20 mm in length. The total length for the first conduit was 12.29 cm. The second conduit (e.g., second conduit 104) coupled between the left atrium and the first conduit had a maximum diameter of 12 mm with a 7.5 mm “nozzle” where it coupled to the narrow segment. The area of the narrow segment was 50% of the area of the aorta (as provided by Equation 16). For Model 2, the diameter of the aorta was 1.46 cm, and the radius of the narrow segment was approximately 0.30 cm.

### Model 3

For Model 3, the first conduit had a first transition segment (e.g., transition segment 132) 3.43 cm long and a second transition segment (e.g., transition segment 134) that was 6.87 cm long. The narrow segment was 20 mm in length. The total length for the first conduit was 12.29 cm. The second conduit (e.g., second conduit 104) coupled between the left atrium and the first conduit had a maximum diameter of 12 mm with a 7.5 mm “nozzle” where it coupled to the narrow segment. The area of the narrow segment was 75% of the area of the aorta (as provided by Equation 16). For Model 3, the diameter of the aorta was 1.46 cm, and the radius of the narrow segment was approximately 0.37 cm.

### Example 2—Performance Evaluation of Models

The Control Model and Models 0-3 of Example 1 were evaluated using SimVascular. For the control and all of the models, the heart rate was set to 60 beats per minute, and blood flow was assumed to be pulsatile flow at the inlet (i.e., ascending aorta).

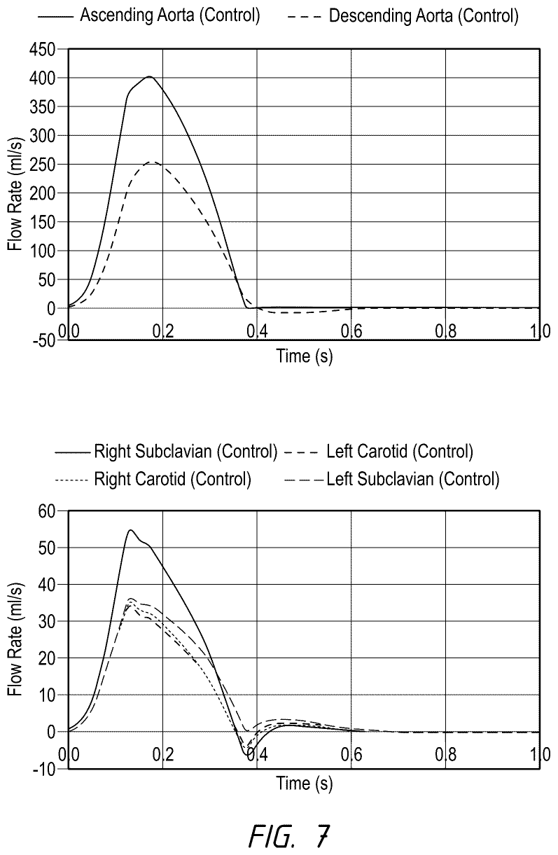

FIG. 7 shows plots of the blood flow rate over time for the Control Model. For the Control Model, the cardiac output (CO) was assumed to be 5 L/min. The flow curves are for data collected at points for the inlet and outlet boundaries during one cardiac cycle. In systole, pressure increased in the aorta, and the blood pressure dropped gradually from the ascending to descending aorta. More uniform pressure distribution was observed in the aorta in diastole. The plots in FIG. 7 indicate blood flow in the ascending aorta was unsteady (pulsatile), and no flow was pumped by the heart in diastole. Pulsatile blood flow in the descending aorta and the aortic arch branches synchronized with the flow in the ascending aorta. Table 1 shows the flow rate magnitude at the inlet and outlet boundaries in the middle of systole. In the middle of systole, 63.6% of the total inflow went through the descending aorta and 36% went through the aortic arch branches. The total volume of blood pumped by the heart in systole was 83.4 ml and in diastole was 0 ml for one cardiac cycle.

TABLE 1
Flow magnitude in middle of systole for Control Model

| | Flow | | Flow | % of total |
|---|---|---|---|---|
| Inflow | rate (ml/s) | Outflow | rate (ml/s) | inflow |
| Ascending | 401.71 | Descending | 255.53 | 63.6% |
| Aorta | | Aorta | | |
| Conduit | — | Right Subclavian | 49.61 | 12.4% |
| Total inflow | 401.71 | Right Carotid | 32.01 | 8.0% |
| | | Left Carotid | 30.34 | 7.6% |
| | | Left Subclavian | 34.22 | 8.5% |

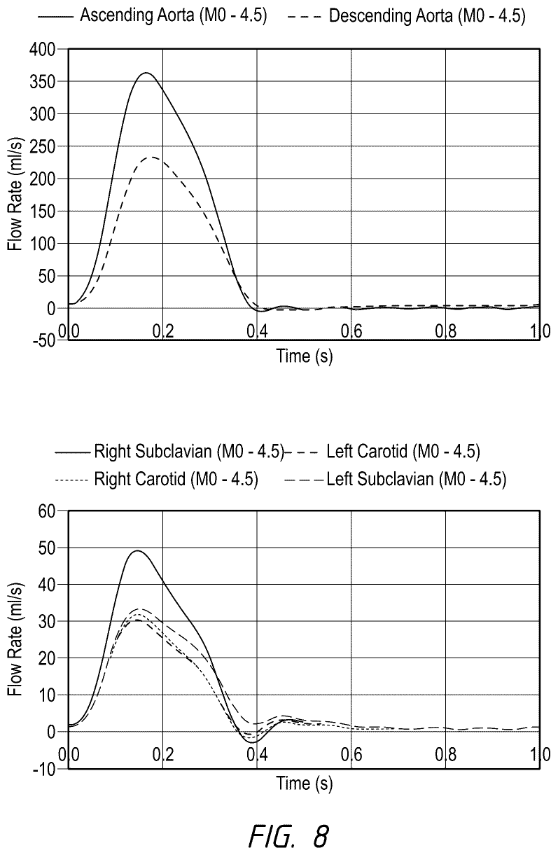

FIG. 8 shows plots of the blood flow rate over time for Model 0 according to an example embodiment of the disclosure. For the plots in FIG. 8, the cardiac output (CO) was assumed to be 4.5 L/min, which is 0.5 L/min less than the control. Flow through the second conduit (between the heart and aorta) of the device was assumed to be 0.5 L/min. In systole, pressure increased in the aorta, and the blood pressure dropped gradually from the ascending to descending aorta. Pressure build-up in the aortic arch was less than in the control case in systole, possibly due to the lower CO. The presence of the device with a constant flow rate of 0.5 L/min changed pressure distribution in the middle of diastole. The flow curves are for data collected at points for the inlet and outlet boundaries during one cardiac cycle. The plots in FIG. 8 indicate blood flow in the ascending aorta was pulsatile, and the device added flow to the system in both systole and in diastole. Table 2 shows the flow rate magnitude at the inlet and outlet boundaries in the middle of systole. In the middle of systole, the flow rate at the ascending aorta was 363.71 ml/s. The device provided a constant flow rate of 8.33 ml/s. 63.4% of the total inflow went through the descending aorta, and 36.6% was pumped through the aortic arch branches. For Model 0 with CO 4.5 L/min, 75 ml of blood was pumped by the heart in systole and zero in diastole, and the device provided 3.1 ml in systole and 5.2 ml in diastole for a total of 83.3 ml for one cardiac cycle.

TABLE 2
Flow magnitude in middle of systole for Model 0 with CO
4.5 L/min and device output of 0.5 L/min

| | Flow | | Flow | % of total |
|---|---|---|---|---|
| Inflow | rate (ml/s) | Outflow | rate (ml/s) | inflow |
| Ascending | 363.71 | Descending | 235.85 | 63.4% |
| Aorta | | Aorta | | |
| Conduit | 8.33 | Right Subclavian | 46.00 | 12.4% |
| Total inflow | 372.04 | Right Carotid | 30.02 | 8.0% |
| | | Left Carotid | 28.24 | 7.6% |
| | | Left Subclavian | 31.93 | 8.6% |

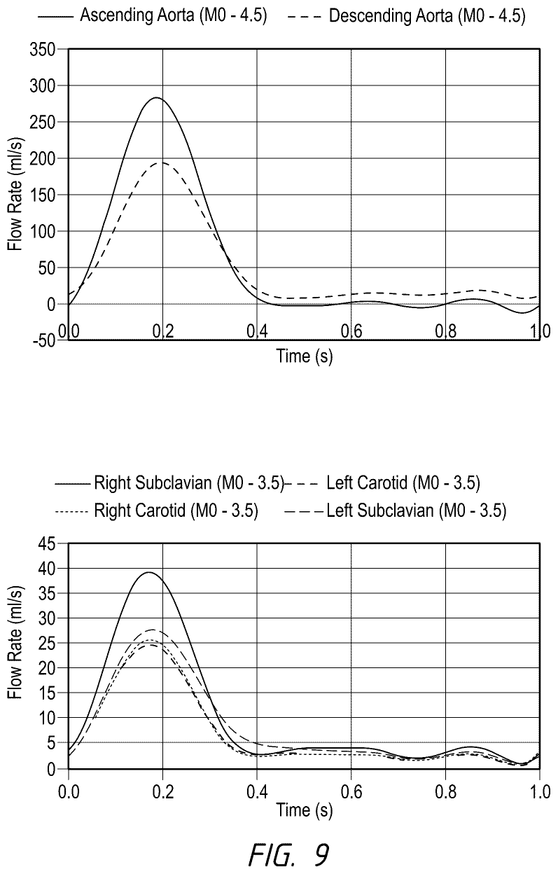

FIG. 9 shows plots of the blood flow rate over time for Model 0 according to an example embodiment of the disclosure. For the plots in FIG. 9, the cardiac output (CO) was assumed to be 3.5 L/min. Flow through the device was assumed to be 1.5 L/min. In systole, pressure increased in the aorta, and the blood pressure dropped gradually from the ascending to descending aorta. Lower pressure was observed in the aortic arch in the middle of systole than in the control case in systole, possibly due to the lower CO. The presence of the second conduit of the device with a constant flow rate of 1.5 L/min changed pressure distribution in the middle of diastole. A local pressure increase was observed at the location of jet impingement (e.g., a location in the narrow portion where blood entered from the second conduit). The flow curves are for data collected at points for the inlet and outlet boundaries during one cardiac cycle. The plots in FIG. 9 indicate blood flow in the ascending aorta was pulsatile, as well as blood flow in the descending aorta and the aortic arch branches, synchronized with the flow in the ascending aorta. The device added flow to the system in both systole and in diastole. Thus, compared to the plots in FIG. 8, the curves in the plots of FIG. 9 are shifted upward. Table 3 shows the flow rate magnitude at the inlet and outlet boundaries in the middle of systole. In the middle of systole, the flow rate at the ascending aorta was 282.77 ml/s. The device provided a constant flow rate of 25 ml/s. 62% of the total inflow went through the descending aorta, and 38% was pumped through the aortic arch branches. For Model 0 with CO 3.5 L/min, 58.3 ml of blood was pumped by the heart in systole and zero in diastole, and the device provided 9.4 ml in systole and 15.6 ml in diastole for a total of 83.3 ml for one cardiac cycle.

TABLE 3
Flow magnitude in middle of systole for Model 0 with CO
3.5 L/min and device output of 1.5 L/min

| | Flow | | Flow | % of total |
|---|---|---|---|---|
| Inflow | rate (ml/s) | Outflow | rate (ml/s) | inflow |
| Ascending | 363.71 | Descending | 235.85 | 63.4% |
| Aorta | | Aorta | | |
| Conduit | 8.33 | Right Subclavian | 46.00 | 12.4% |
| Total inflow | 372.04 | Right Carotid | 30.02 | 8.0% |
| | | Left Carotid | 28.24 | 7.6% |
| | | Left Subclavian | 31.93 | 8.6% |

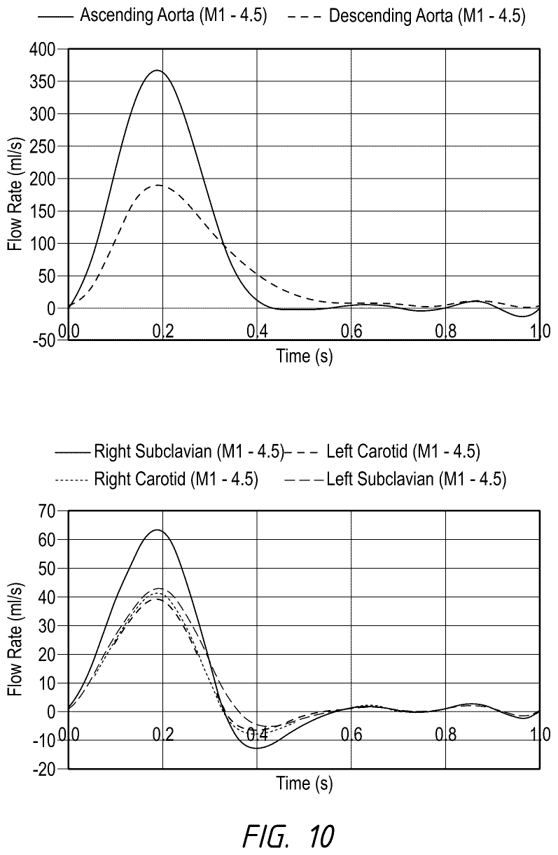

FIG. 10 shows plots of the blood flow rate over time for Model 1 according to an example embodiment of the disclosure. For the plots in FIG. 10, the cardiac output (CO) was assumed to be 4.5 L/min. Flow through the second conduit of the device was assumed to be 0.5 L/min. For Model 1, pressure increased during systole in the ascending aorta and the branches of the aortic arch. A pressure difference was also observed before and after the narrow segment of the device in the descending aorta. In diastole, the device of Model 1 altered the pressure distribution in the aorta compared to the Control Model. The flow curves are for data collected at points for the inlet and outlet boundaries during one cardiac cycle. The plots in FIG. 10 indicate blood flow in the ascending aorta was pulsatile, and the device added flow to the system in both systole and in diastole. Table 4 shows the flow rate magnitude at the inlet and outlet boundaries in the middle of systole. In the middle of systole, the flow rate at the ascending aorta was 363.56 ml/s. The device provided a constant flow rate of 8.33 ml/s. 50.1% of the total inflow went through the descending aorta, and 49.9% was pumped through the aortic arch branches. For Model 1 with CO 4.5 L/min, 75 ml of blood was pumped by the heart in systole and zero in diastole, and the device provided 3.1 ml in systole and 5.2 ml in diastole for a total of 83.3 ml for one cardiac cycle.

TABLE 4
Flow magnitude in middle of systole for Model 1 with CO 4.5
L/min and device output of 0.5 L/min

| | Flow | | Flow | % of total |
|---|---|---|---|---|
| Inflow | rate (ml/s) | Outflow | rate (ml/s) | inflow |
| Ascending | 363.71 | Descending | 235.85 | 63.4% |
| Aorta | | Aorta | | |
| Conduit | 8.33 | Right Subclavian | 46.00 | 12.4% |
| Total inflow | 372.04 | Right Carotid | 30.02 | 8.0% |
| | | Left Carotid | 28.24 | 7.6% |
| | | Left Subclavian | 31.93 | 8.6% |

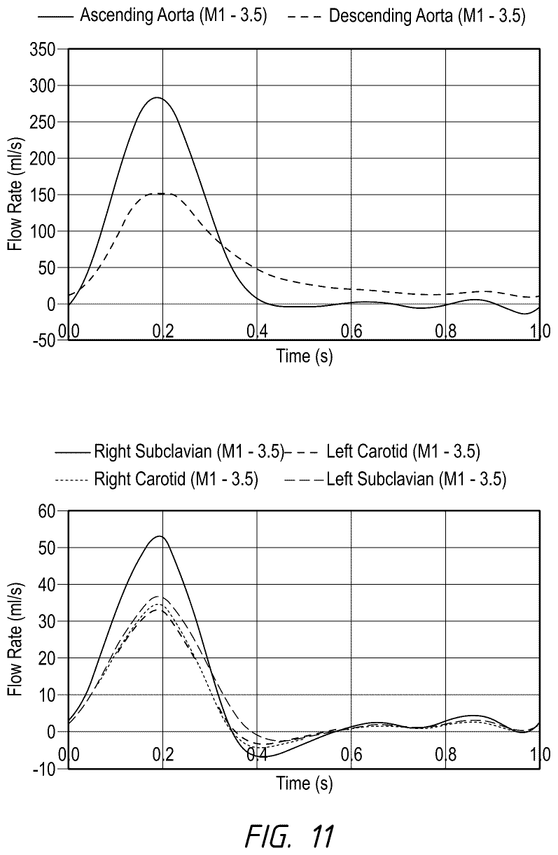

FIG. 11 shows plots of the blood flow rate over time for Model 1 according to an example embodiment of the disclosure. For the plots in FIG. 11, the cardiac output (CO) was assumed to be 3.5 L/min. Flow through the second conduit of the device was assumed to be 1.5 L/min. In systole, pressure increased in the ascending aorta and branches of the aortic arch. Considering that the cardiac output was 3.5 L/min, the pressure increase in the aortic arch was less than Model 1 with a cardiac output of 4.5 L/min. In the middle of systole, a pressure difference was observed before and after the narrow segment in the descending aorta. In diastole, the device altered the pressure distribution in the aorta compared to the Control Model. A local pressure increase was observed at the location of jet impingement (e.g., a location in the narrow portion where blood entered from the second conduit). The flow curves are for data collected at points for the inlet and outlet boundaries during one cardiac cycle. The plots in FIG. 11 indicate blood flow in the ascending aorta was pulsatile, as well as blood flow in the descending aorta and the aortic arch branches, but the shape was different than in the previous examples. The device added flow to the system in both systole and in diastole. Thus, compared to the previous plots, the curves in the plots of FIG. 11 are shifted upward. Table 5 shows the flow rate magnitude at the inlet and outlet boundaries in the middle of systole. In the middle of systole, the flow rate at the ascending aorta was 282.77 ml/s. The device provided a constant flow rate of 25 ml/s. 49.4% of the total inflow went through the descending aorta, and 50.6% was pumped through the aortic arch branches. For Model 1 with CO 4.5 L/min, 58.3 ml of blood was pumped by the heart in systole and zero in diastole, and the device provided 9.4 ml in systole and 15.6 ml in diastole for a total of 83.3 ml for one cardiac cycle.

TABLE 5
Flow magnitude in middle of systole for Model 1 with CO 3.5
L/min and device output of 1.5 L/min

| | Flow | | Flow | % of total |
|---|---|---|---|---|
| Inflow | rate (ml/s) | Outflow | rate (ml/s) | inflow |
| Ascending | 282.77 | Descending | 152.08 | 49.4 |
| Aorta | | Aorta | | |
| Conduit | 25.00 | Right Subclavian | 52.31 | 17.0 |
| Total inflow | 307.77 | Right Carotid | 34.44 | 11.2 |
| | | Left Carotid | 32.78 | 10.7 |
| | | Left Subclavian | 36.16 | 11.7 |

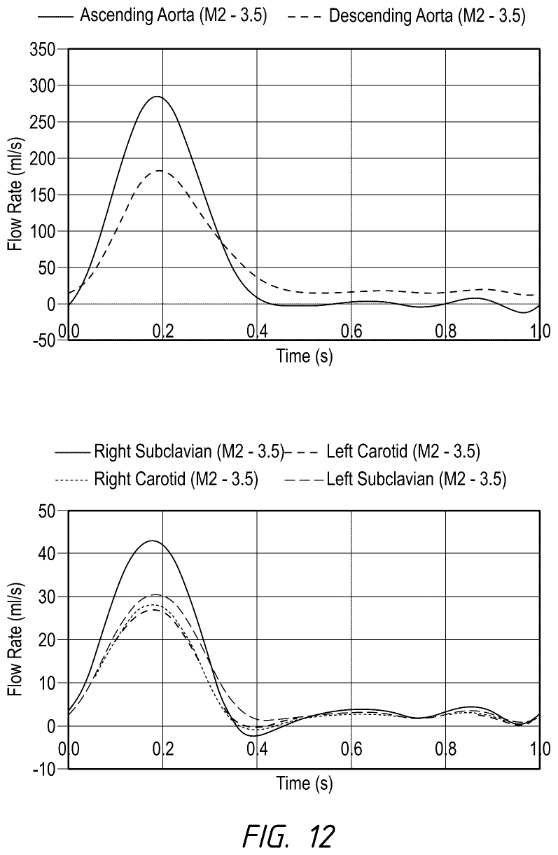

FIG. 12 shows plots of the blood flow rate over time for Model 2 according to an example embodiment of the disclosure. For the plots in FIG. 12, the cardiac output (CO) was assumed to be 3.5 L/min. Flow through the second conduit of the device was assumed to be 1.5 L/min. For Model 2, pressure increased during systole in the ascending aorta and the branches of the aortic arch, but the pressure increase was less than Model 1 at a CO of 3.5 L/min and a device flow of 1.5 L/min. A pressure difference was also observed before and after the narrow segment of the device in the descending aorta. In diastole, the device in Model 2 altered the pressure distribution in the aorta compared to the Control Model. A local pressure increase was observed at the location of jet impingement (e.g., a location in the narrow portion where blood entered from the second conduit). The flow curves are for data collected at points for the inlet and outlet boundaries during one cardiac cycle. The plots in FIG. 12 indicate blood flow in the ascending aorta was pulsatile, as well as the flow in the descending aorta and aortic arch branches. The device added flow to the system in both systole and in diastole. Table 6 shows the flow rate magnitude at the inlet and outlet boundaries in the middle of systole. In the middle of systole, the flow rate at the ascending aorta was 282.77 ml/s. The device provided a constant flow rate of 25 ml/s. 58.2% of the total inflow went through the descending aorta, and 41.8% was pumped through the aortic arch branches. For Model 2 with CO 3.5 L/min, 58.3 ml of blood was pumped by the heart in systole and zero in diastole, and the device provided 9.4 ml in systole and 15.6 ml in diastole for a total of 83.3 ml for one cardiac cycle.

TABLE 6
Flow magnitude in middle of systole for Model 2 with CO 3.5
L/min and device output of 1.5 L/min

| | Flow | | Flow | % of total |
|---|---|---|---|---|
| Inflow | rate (ml/s) | Outflow | rate (ml/s) | inflow |
| Ascending | 282.77 | Descending | 179.17 | 58.2% |
| Aorta | | Aorta | | |
| Conduit | 25.00 | Right Subclavian | 43.10 | 14.0% |
| Total inflow | 307.77 | Right Carotid | 28.21 | 9.2% |
| | | Left Carotid | 26.99 | 8.8% |
| | | Left Subclavian | 30.29 | 9.8% |

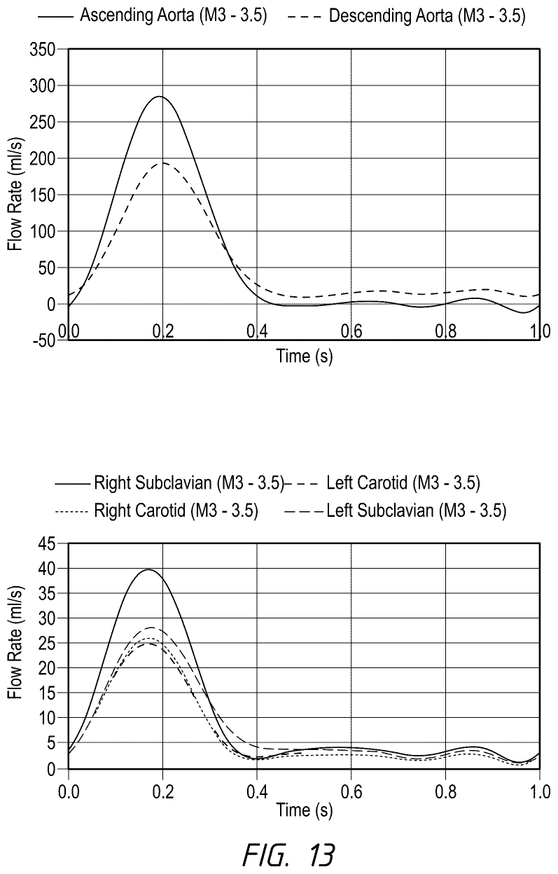

FIG. 13 shows plots of the blood flow rate over time for Model 3 according to an example embodiment of the disclosure. For the plots in FIG. 13, the cardiac output (CO) was assumed to be 3.5 L/min. Flow through the second conduit of the device was assumed to be 1.5 L/min. In systole, pressure increased in the ascending aorta and branches of the aortic arch compared to the control, but the pressure increase in the aortic arch was less than Models 1 and 2 at the same CO and device flow. In the middle of systole, a pressure difference was observed before and after the narrow segment in the descending aorta. In diastole, the device altered the pressure distribution in the aorta compared to the control model. A local pressure increase was observed at the location of jet impingement (e.g., a location in the narrow portion where blood entered from the second conduit). The flow curves are for data collected at points for the inlet and outlet boundaries during one cardiac cycle. The plots in FIG. 13 indicate blood flow in the ascending aorta was pulsatile, as well as blood flow in the descending aorta and the aortic arch branches. The device added flow to the system in both systole and in diastole. Table 7 shows the flow rate magnitude at the inlet and outlet boundaries in the middle of systole. In the middle of systole, the flow rate at the ascending aorta was 282.77 ml/s. The device provided a constant flow rate of 25 ml/s. 61.3% of the total inflow went through the descending aorta, and 38.7% was pumped through the aortic arch branches. For Model 3 with CO 3.5 L/min, 58.3 ml of blood was pumped by the heart in systole and zero in diastole, and the device provided 9.4 ml in systole and 15.6 ml in diastole for a total of 83.3 ml for one cardiac cycle.

TABLE 7
Flow magnitude in middle of systole for Model 3 with CO 3.5
L/min and device output of 1.5 L/min

| | Flow | | Flow | % of total |
|---|---|---|---|---|
| Inflow | rate (ml/s) | Outflow | rate (ml/s) | inflow |
| Ascending | 282.77 | Descending | 188.69 | 61.3% |
| Aorta | | Aorta | | |
| Conduit | 25.00 | Right Subclavian | 39.89 | 13.0% |
| Total inflow | 307.77 | Right Carotid | 26.05 | 8.5% |
| | | Left Carotid | 24.97 | 8.1% |
| | | Left Subclavian | 28.16 | 9.1% |

### Example 3—Comparisons of Simulated Flow Rates

The simulated flow rates of Example 2 were compared within and between the Control Model and Models 0-3 of Example 1.

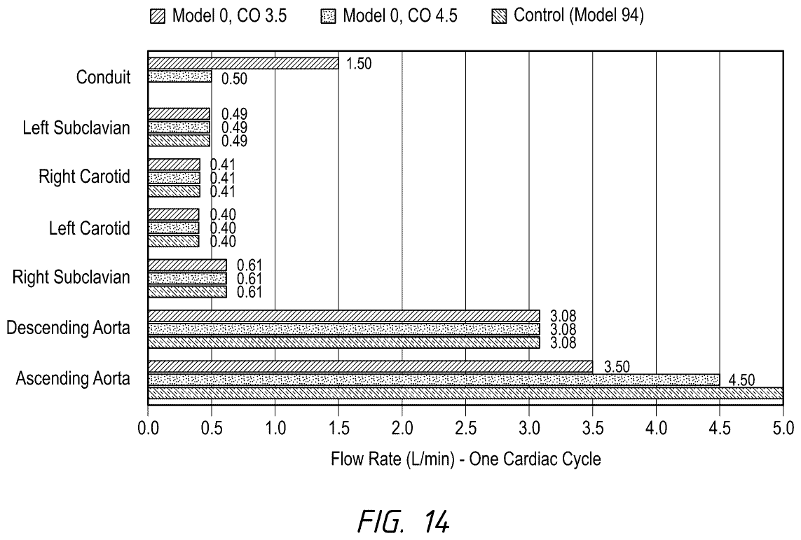

FIG. 14 is a chart comparing the performance of the Model 0 example to the Control Model. The chart illustrates a side-by-side comparison of Model 0 to the Control Model based on the “total” flow rate passed through the boundaries in one cardiac cycle. The results shown in FIG. 14 indicate the device compensated for reduced blood flow in the descending aorta and the aortic arch branches when cardiac output was decreased (e.g., from 5.0 L/ml in the Control Model to 4.5 L/ml and 3.5 L/ml simulated for Model 0).

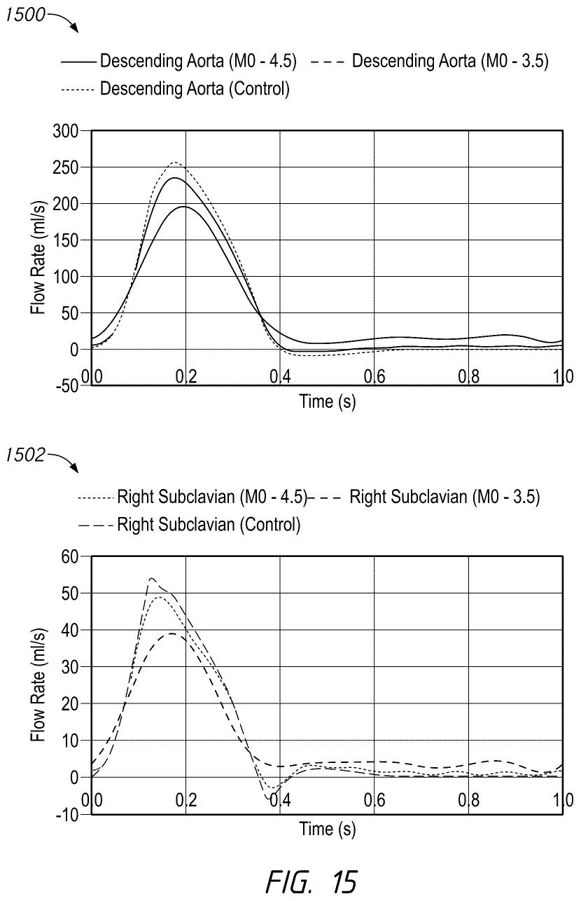

Between the two flow rates simulated for Model 0, variations in flow rate during a cardiac cycle were not similar. FIG. 15 shows flow rates over time during one cardiac cycle for three cardiac outputs according to embodiments of the present disclosure. As indicated in plot 1500, flow rate variations in the descending aorta were observed. In systole, the highest flow rate was observed in the control case with a CO of 5 L/min. Plot 1500 shows a higher maximum flow rate in the descending aorta in Model 0 with a CO of 4.5 L/min than a CO of 3.5 L/min in systole. In diastole, however, the trend was reversed in the descending aorta. A higher flow rate magnitude in the descending aorta with the device was observed. The higher the device's flow rate, the higher the flow rate was in the descending aorta in diastole.

A similar trend was observed in aortic arch branches. Plot 1502 shows the flow rate variations during one cardiac cycle in the right subclavian artery. In systole, flow going through the right subclavian artery was lower in Model 0 with a CO of 3.5 L/min than in Model 0 with a CO of 4.5 L/min. In diastole, however, flow going through the right subclavian artery was higher in Model 0 with a CO of 3.5 L/min than in Model 0 with a CO of 4.5 L/min. Early in systole, in the presence of the device, the flow rate was not zero. The device also reduced the negative flow rate observed early in diastole. Similar behavior was observed in other aortic arch branches as illustrated by data for the right subclavian artery shown in plot 1502 (data for other branches not shown).

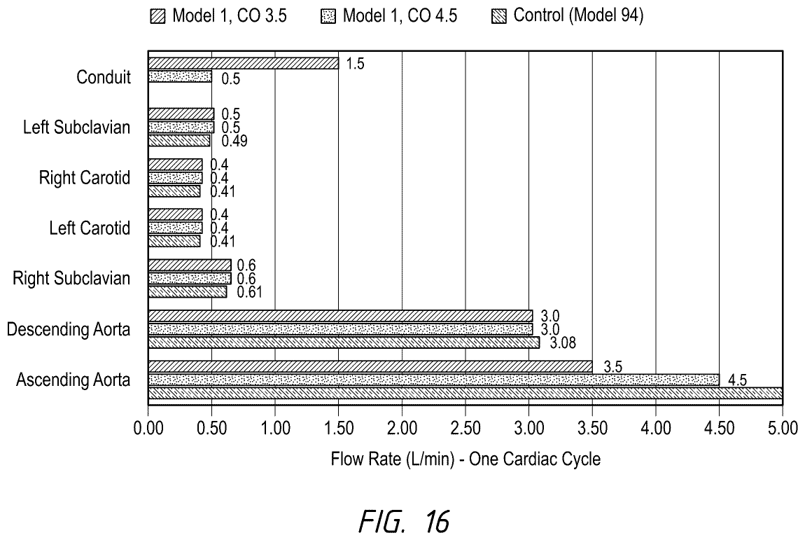

FIG. 16 is a chart comparing the performance of the Model 1 example to the Control Model. The chart shows a side-by-side comparison of Model 1 to the Control Model based on the “total” flow rate passed through the boundaries in one cardiac cycle. The results shown in FIG. 16 indicate the device compensated for reduced blood flow in the descending aorta and the aortic arch branches when cardiac output was decreased (e.g., from 5.0 L/ml in the Control Model to 4.5 L/ml and 3.5 L/ml simulated for Model 1).

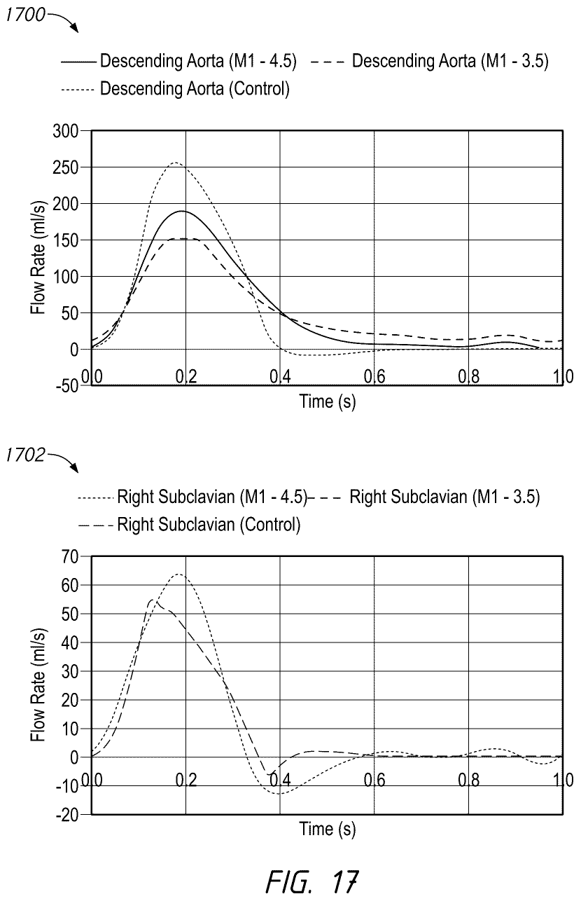

Between the two flow rates simulated for Model 1, variations in flow rate during a cardiac cycle were not similar. FIG. 17 shows flow rates over time during one cardiac cycle for three cardiac outputs according to embodiments of the present disclosure. As indicated in plot 1500, in systole, the highest flow rate was observed in the control case with a CO of 5 L/min. A higher maximum flow rate in Model 1 with a CO of 4.5 L/min than a CO of 3.5 L/min in systole was observed. In diastole, however, the trend was reversed. A higher flow rate magnitude in the descending aorta with the device was observed. The higher the device's flow rate, the higher the flow rate was in the descending aorta in diastole. A similar trend was observed in aortic arch branches. Plot 1702 shows the flow rate variations during one cardiac cycle in the right subclavian artery. In systole, flow going through the right subclavian artery was lower in Model 1 with a CO of 3.5 L/min than in Model 1 with a CO of 4.5 L/min. In diastole, however, flow going through the right subclavian artery was higher in Model 1 with a CO of 3.5 L/min than in Model 1 with a CO of 4.5 L/min. Early in systole, in the presence of the device, the flow rate was not zero. The device also reduced the negative flow rate observed early in the diastole. Similar behavior was observed in other aortic arch branches (data not shown).

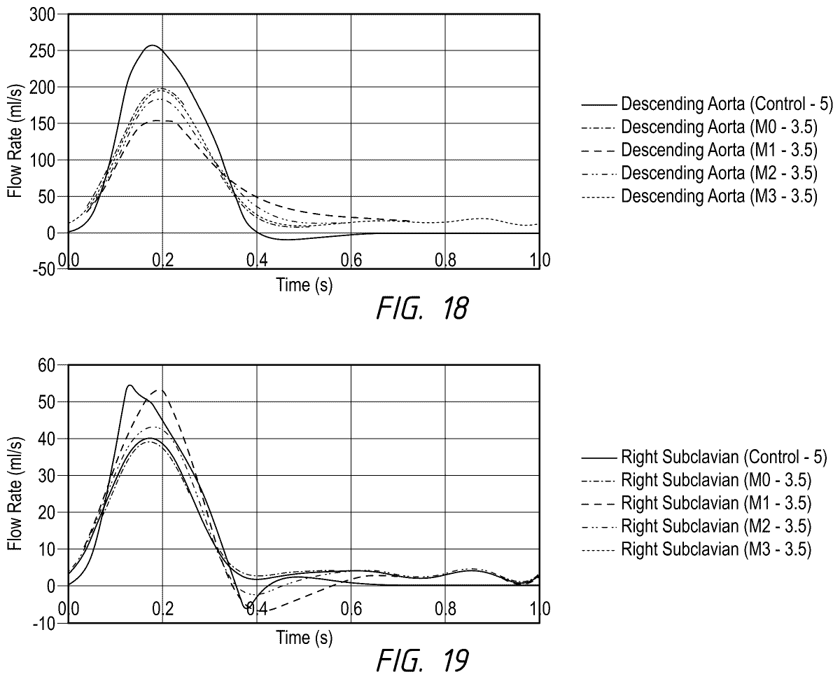

FIG. 18 is a plot of flow rates over time for one cardiac cycle in the descending aorta for a fixed cardiac output according to examples of the present disclosure. Models 0, 1, 2, and 3 had a fixed cardiac output of 3.5 L/min. In systole, the highest flow rate was observed in the control case with a CO of 5 L/min. In Model 0, the maximum flow rate in systole dropped compared to the control because of the reduced cardiac output of 3.5 L/min versus the cardiac output of 5 L/min of the control. The presence of the device in the descending aorta further reduced the maximum flow rate in the descending aorta in systole. As the diameter of the narrow segment increased, a higher flow rate was observed in the descending aorta in systole. Early in diastole, the trend was reversed. In Models 0, 1, 2, and 3, the device added flow to the system in systole and diastole. The flow curves in diastole were shifted upward compared to the control case.

FIG. 19 is a plot of flow rates over time for one cardiac cycle in the subclavian artery for a fixed cardiac output according to examples of the present disclosure. Models 0, 1, 2, and 3 had a fixed cardiac output of 3.5 L/min. In systole, the highest flow rate was observed in the control case with a cardiac output of 5 L/min. In Model 0, the maximum flow rate in systole dropped significantly because of the reduced cardiac output of 3.5 L/min. The presence of a narrow segment in Models 1-3 in the descending aorta increased the maximum flow rate in the right subclavian artery in systole. As the diameter of the narrow segment increased, a lower flow rate was observed in the right subclavian artery in systole. In diastole, the device shifted the flow curves upward in Models 0, 1, 2, and 3.

### Example 4—Pressure Contours

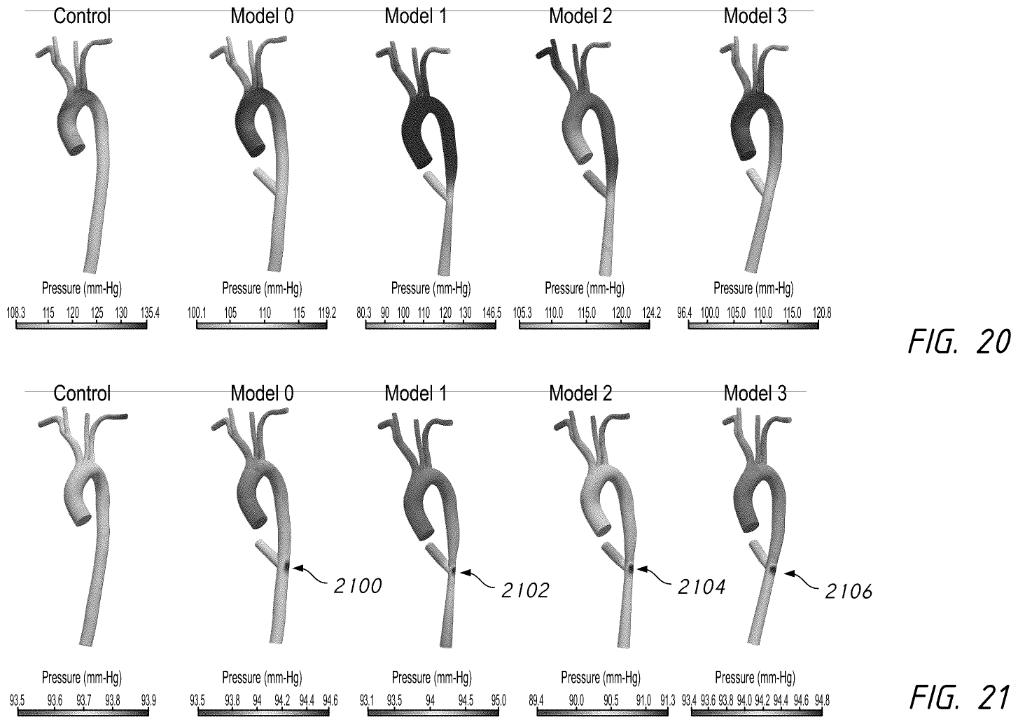

FIG. 20 shows pressure contours of the control and the example devices in middle systole according to embodiments of the present disclosure. The pressure distribution for the Control Model is for a cardiac output of 5 L/min and the pressure distributions in Models 0, 1, 2, and 3 are for a cardiac output of 3.5 L/min. The maximum pressure in the Control Model was 135.4 mm-Hg. In Model 0, with the reduced cardiac output, the maximum pressure was 119.2 mm-Hg. Adding a narrow segment to the device increased blood pressure in the aortic arch. The maximum pressure in Models 1, 2, and 3 was 146.5 mm-Hg, 124.2 mm-Hg, and 120.8 mm-Hg, respectively. As noted previously, a 12 mm conduit connected the left atrium to the descending aorta in Model 0. In Models 1, 2, and 3, a conical nozzle shape was provided on the end of the second conduit coupled to the first conduit to facilitate connection to the narrow portion. The form of the second conduit was identical in Models 1, 2, and 3. Pressure at the inlet of the conduit in the middle of systole was 108.8 mmHg, 115.6 mmHg, 105.7 mmHg, and 107.9 mmHg in Models 0, 1, 2, and 3, respectively. In Model 1, pressure dropped in the narrow segment, but because of the elevated pressure in the arch, the actual pressure in the narrow segment was more than Model 0. In Model 2 and Model 3, pressure at the inlet of the conduit was lower than in Model 0. The data indicate that the device may consume slightly less energy under these conditions than Model 0 in systole, which may be due in part to the presence of the narrow segment.

FIG. 21 shows pressure contours of the control and the example devices in middle diastole according to embodiments of the present disclosure. The pressure distribution for the Control Model is for a cardiac output of 5 L/min and the pressure distributions in Models 0, 1, 2, and 3 are for a cardiac output of 3.5 L/min. In Model 0, because of the presence of the device, slightly higher pressure was observed in the descending aorta compared to the control case. In the middle of diastole, the pressure was 94.0 mmHg, 94.4 mmHg, 94.4 mmHg, and 94.3 mmHg at the inlet of the second conduit in Models 0, 1, 2, and 3, respectively. The pressure values indicate the device with and without the narrow segment will consume a similar energy level in the diastole. A local pressure increase was observed in Models 0, 1, 2, and 3 at the location ofjet impingement as indicated by arrows 2100, 2102, 2104, and 2106, respectively.

The simulation results for the example devices indicate that the devices as disclosed herein may compensate for the reduced blood flow in the descending aorta and the aortic arch branches during a cardiac cycle. The simulation results also showed that placing a narrow segment in the descending aorta may increase pressure in the aortic arch in systole. If the ratio of the narrow segment area to the descending aorta area ratio is less than 50% (e.g., Model 1), because of the elevated pressure in the aortic arch, the actual pressure in the narrow segment could be more than when no narrow segment is present. A narrow segment area to descending aorta area ratio equal to or more than 50% (Model 2 and Model 3), however, may reduce the energy consumption of the device during systole. The simulations showed that the example devices with and without the narrow segment may consume a similar energy level in the diastole. Thus, the presence of a device as disclosed herein may alter blood flow hemodynamics (e.g., pressure and flow distributions) in descending aorta and aortic arch branches and may compensate, at least in part, for reduced cardiac output.

As used herein, the terms “about” and “same” (which, as used herein, may mean, e.g., equal, substantially equal) modifying, for example, the length of a component, a diameter of a component, a volume, a flow rate through at least a portion of a component, and ranges thereof, employed in describing the embodiments of the disclosure, refers to variation in the numerical quantity that can occur, for example, through typical measuring and manufacturing procedures used for making devices; through inadvertent error in these procedures; through differences in the manufacture, implantation, or installation techniques used to provide the devices or carry out the methods, and like proximate considerations. In some instances, the terms “about” and “same” include values up to and including 10% less than and 10% greater than the recited value.

Of course, it is to be appreciated that any one of the examples, embodiments or processes described herein may be combined with one or more other examples, embodiments and/or processes or be separated and/or performed amongst separate devices or device portions in accordance with the present systems, devices and methods.

Finally, the above-discussion is intended to be merely illustrative of the present system and should not be construed as limiting the appended claims to any particular embodiment or group of embodiments. Thus, while the present system has been described in particular detail with reference to exemplary embodiments, it should also be appreciated that numerous modifications and alternative embodiments may be devised by those having ordinary skill in the art without departing from the broader and intended spirit and scope of the present system as set forth in the claims that follow. Accordingly, the specification and drawings are to be regarded in an illustrative manner and are not intended to limit the scope of the appended claims.

1. A device for delivering blood from a lower pressure region to a higher pressure region, the device comprising:
a first conduit configured to be disposed within the higher pressure region, comprising:
a first end having a first diameter;
a second end opposite the first end and having a second diameter; and
a narrow segment disposed between the first end and the second end and having a third diameter;
a second conduit comprising:
a third end configured to be fluidly coupled to the lower pressure region; and
a fourth end opposite the third end, wherein the fourth end is coupled to the first conduit at or near the narrow segment of the first conduit; and
a pump disposed along the second conduit between the third end and the fourth end.

2. The device of claim 1, further comprising a power source coupled to the pump.

3. The device of claim 2, wherein the power source comprises a transcutaneous energy transfer (TET) system.

4. The device of claim 3, wherein the TET system comprises:
an internal TET controller and telemetry system;
an external TET controller and telemetry system;
a primary coil coupled to the external TET controller and telemetry system;
a secondary coil coupled to the internal TET controller and telemetry system; and
a battery pack coupled to the external TET controller and telemetry system.

5. The device of claim 1, wherein the pump comprises an axial flow pump, a peristaltic pump, or a combination thereof.

6. The device of claim 1, wherein the pump is programmable to set a pumping rate, a flow type, or a combination thereof.

7. The device of claim 6, wherein the flow type comprises continuous flow, variable flow, pulsatile flow, or a combination thereof.

8. The device of claim 6, wherein the pumping rate is programmable from 0.5-2.5 liters per minute.

9. The device of claim 1, wherein the third diameter is less than the first diameter and the second diameter.

10. The device of claim 1, wherein an area of the narrow conduit is 35-75% of an area of the first end, wherein the area of the narrow conduit is calculated from the third diameter and the area of the first end is calculated from the first diameter.

11. The device of claim 1, wherein the third diameter is equal to the first diameter.

12. The device of claim 1, wherein the second conduit has a fourth diameter and comprises a nozzle at the fourth end, wherein an end of the nozzle coupled to the fourth end has a fifth diameter less than the fourth diameter.

13. The device of claim 12, wherein the fourth diameter is about 12 millimeters (mm) and the fifth diameter is about 7.5 mm.

14. A method of delivering blood from a lower pressure region to a higher pressure region, the method comprising:
flowing, through a first conduit disposed in the higher pressure region, a first portion of blood from the higher pressure region, wherein a first end of the first conduit is coupled to the higher pressure region at a first location and a second end of the first conduit is coupled to the higher pressure region at a second location downstream of the first location;
diverting, through a second conduit, a second portion of blood from the lower pressure region, wherein a third end of the second conduit is fluidly coupled to the lower pressure region and a fourth end of the second conduit is fluidly coupled to a narrow segment of the first conduit, the narrow segment disposed between the first end and the second end;
drawing the second portion of blood from the second conduit into the first conduit, at least in part, by pumping, with a pump, the second portion of blood along the second conduit; and
providing the first portion of blood and the second portion of blood to the higher pressure region at the second end of the first conduit.

15. The method of claim 14, wherein the lower pressure region comprises a left atrium of a heart and the higher pressure region comprises an aorta.

16. The method of claim 14, wherein drawing the second portion of blood from the second conduit into the first conduit further comprises generating, with the narrow segment, a Venturi effect.

17. The method of claim 14, further comprising programming the pump to set a pumping rate, a flow type, or a combination thereof.

18. The method of claim 17, wherein the flow type comprises continuous flow, variable flow, pulsatile flow, or a combination thereof.

19. The device of claim 14, wherein the pump comprises an axial flow pump, a peristaltic pump, or a combination thereof.

20. The method of claim 14, further comprising powering the pump with a power source comprising a transcutaneous energy transfer (TET) system.

21. (canceled)

22. (canceled)

23. (canceled)
